(19) United States
(12) Patent Application Publication
(10) Pub. No.: US 2021/0402380 A1
(43) Pub. Date: Dec. 30, 2021

(54) PEROVSKITES FOR PHOTOCATALYTIC ORGANIC SYNTHESIS

(71) Applicant: San Diego State University Foundation, San Diego, CA (US)

(72) Inventor: Yong YAN, San Diego, CA (US)

(73) Assignee: San Diego State University Foundation, San Diego, CA (US)

(21) Appl. No.: 17/350,431

(22) Filed: Jun. 17, 2021

Related U.S. Application Data
(60) Provisional application No. 63/042,706, filed on Jun. 23, 2020.

(51) Int. Cl. B01J 27/135; B01J 31/12; B01J 31/30; B01J 27/128; B01J 35/00; B01J 35/02; B01J 19/12; C07C 45/72; C07D 217/16; C07D 498/04; C07D 231/12; C07D 207/34; C07C 67/10; C07D 217/12; C07D 413/12; C07D 211/94; C07D 405/04; C07D 211/90

(52) U.S. Cl. CPC B01J 27/135; C07C 2601/16; B01J 31/30; B01J 27/128; B01J 35/004; B01J 35/0013; B01J 35/026; B01J 35/0006; B01J 19/127; B01J 19/123; C07C 45/72; C07D 217/16; C07D 498/04; C07D 231/12; C07D 207/34; C07C 67/10; C07D 217/12; C07D 413/12; C07D 211/94; C07D 405/04; C07D 211/90; B01J 2531/007; B01J 2231/4277; B01J 2231/46; B01J 2231/326; B01J 2231/49; B01J 2219/1203; B01J 2219/0892; B01J 31/127

(57) ABSTRACT

Nature is capable of storing solar energy in chemical bonds via photosynthesis through a series of C—C, C—O and C—N bond-forming reactions starting from CO₂ and light. Direct capture of solar energy for organic synthesis is a promising approach. Lead (Pb)-halide perovskite solar cells reach 24.2% power conversion efficiency, rendering perovskite a unique type material for solar energy capture. We show that photophysical properties of perovskites is useful in photoredox organic synthesis. Because the key aspects of these two applications are both relying on charge separation and transfer. Here we demonstrated that perovskites nanocrystals are exceptional candidates as photocatalysts for fundamental organic reactions, i.e. C—C, C—N and C—O bond-formations. Stability of CsPbBr₃ in organic solvents and ease-of-tuning their bandedges garner perovskite a wider scope of organic substrate activations.

### RELATED APPLICATIONS

This application claims priority under 35 U.S.C. § 119(e) to U.S. Provisional Patent Application No. 63/042,706, filed Jun. 23, 2020, which is incorporated herein by reference.

### GOVERNMENT SUPPORT

This invention was made with government support under Grant No. 1851747 awarded by the National Science Foundation and Grant No. DE-AC36-08G028308 awarded by the Department of Energy. The government has certain rights in the invention.

### BACKGROUND OF THE INVENTION

The intentional construction of organic compounds via cost-effective and efficient photocatalysis is highly desirable. Remarkable advances in artificial C—C, C—O and C—N bond formations have been made, including the development of protocols to merge photoredox catalysis with organic, transition metal catalysis and inorganic semiconductors. However, many current catalysts require: high-cost noble metals; complicated synthetic preparations; air-free reaction conditions; or demonstrate moderate activity and are thus, not desirable.

Of the potential photoactive materials, Pb-halide perovskites APbBr₃ are an attractive candidate. They have shown promise for low cost solar energy conversion (e.g., they have strong light absorption, long excited state lifetimes, efficient separation and transport of opposite charge carriers). As a result, revolutionary advances have been claimed in perovskite photovoltaics, i.e. PCE has reached greater than 24.2% in only a few years of development.

Given the widespread success of perovskite in both efficient charge separation and electron-hole diffusion (length>175 μm), we recently questioned whether it might be possible to apply this unique material that has been proved in photovoltaics towards highly efficient photocatalytic organic synthesis. In photovoltaics, the absorption of photons induces the creation of electron/holes, while in photocatalysis, the equivalent is the production of reducing/oxidizing charges that can drive the desired chemistry. For photocatalysis, such reducing/oxidizing equivalents (excited electrons/holes) should live long enough and be transported efficiently to a catalytic site where chemistry occurs (i.e., at the photocatalyst surface). Therefore, photophysical properties of Pb-halide perovskites demonstrated for photovoltaic applications, also should be of interest in photocatalytic organic synthesis. We recently demonstrated that the intrinsic surfaces of MAPbI₃ and MAPbBr₃ perovskites have low surface recombination velocities indicative of an intrinsic low surface defect density that would otherwise hinder surface chemical reactions needed for photocatalytic systems. Our initial exploration of perovskite towards photocatalytic α-alkylation of aldehydes successfully proved that C—C bond formation reactions are efficiently achievable. Other organic reactions focusing on styrene polymerization, benzenethiol dimerization and C—P bond formation between tertiary amines and phosphite esters were also reported. We also note that a perovskite based photocatalyst cell, perovskite/TiO₂ or NiOₓ/perovskite/TiO₂ is report to photooxidize benzylic alcohol or activate C(sp³)-H bond, although the yield is low, ranged from 0.016% to 0.73%. Presently, it is still unknown if perovskites can make a general impact on organic synthesis.

Accordingly. there remains a significant challenge to develop a much needed and easy-to-produce, economical, effective and highly-tolerant photocatalyst for a broad scope of chemical bond formations.

### SUMMARY

This disclosure provides C—C, C—O and C—N bond formations that are of fundamental significance in drug development and materials synthesis, which are realized via perovskite nanocrystals (NCs) in high yield under visible light. Perovskites' unique role towards charge separation and transfer in photocatalytic reactions has been illustrated. Key concerns on perovskite as a photocatalyst, i.e. size, stability, reaction condition tolerance and key catalytic metrics have been discussed. Moreover, band-tuning of perovskite using halide-exchange has been experimentally employed to activate previously unachievable reactions.

Accordingly, this disclosure provides a method for photo-catalytic synthesis of an organic molecule comprising:
a) contacting a lead halide perovskite, first redox substrate, second redox substrate, and solvent to form a mixture; and
b) irradiating the mixture at a suitable wavelength to form an organic molecule having at least one covalent bond between the first redox substrate and second redox substrate, wherein the perovskite photo-catalyzes formation of the at least one covalent bond;

wherein the organic molecule is thereby photo-catalytically synthesized.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Characterization and spectroscopy studies of photocatalysts. (a) TEM of CsPbBr₃ P1; (b) P3; (c) P4; (d) UV-vis and PL spectra of CsPbBr₃ P2-P5; (e) PL spectra for P1 and P4 in CH₂Cl₂ as prepared and after LED irradiation for 24 h and 1 h, respectively. (f) XRD of as-prepared CsPbBr₃ P1; isolated from the reaction 1a before and after irradiation, respectively; (g) the corresponding XRD for reaction 1b; (h) PL spectra of P1 in THF with addition of TFA; (i) PL spectra of CsPbBr₃ NCs, Ir(ppy)₃, CdSe QDs and Ru(bpy)₃Cl₂ in air or N₂-saturated solutions.

FIG. 2. Band energy of CsPbBr₃ vs the redox potentials of substrates.

FIG. 3. Band-tuning of perovskite (Scheme 4). (a) The PL spectra of colloidal CsPbBr₃ in dichloromethane via band tuning with trimethylsilyl chloride or iodide and their representative images under UV lamp (top). (b) Bandedges of APbClxBryI3-x-y; (c) Excited-state potential (E*) range of APbClxBryI3-x-y comparing with noble transition-metal catalysts.

FIG. 4. The demonstration of CsPbBr₃ nanocrystals P1 synthesis.

FIG. 5. The PL spectra for as-prepared CsPbBr₃ P4 and after 8 weeks in Hexane. Inset: the photograph of the sample after 8 weeks (left) and as-prepared (right) in Hexane under the irradiation of 365 nm UV light (right).

FIG. 6. The comparation yields for the synthesis of 1a and 2a by using different perovskites. (a) Time dependence for the perovskite P1-P5 of reaction yields for 1a; (b) Time dependence for the perovskite P1, P2 and P4 of reaction yields for 2a; Reusability test for the perovskite P1, and P4, of reaction yields for 1a (c) and 2a (d). Yield determined by ¹H NMR.

FIG. 7. The photograph for CsPbBr₃ P1 disperse in different solvents. Left, less polar solvents: Hexane, Toluene, 1,4-dioxane, ethyl acetate; right, polar solvents: Acetone, acetonitrile, DMF, and DMSO at ambient light (top) and 365 nm UV light (bottom).

FIG. 8. The comparison of P4 in halide or non-halide solvents. (a) The photograph for CsPbBr₃ P4 before and after the irradiation of blue LED for 2 h in Hexane, CH₂Cl₂ and CH₂Br₂ at ambient light (top) and 365 nm UV light (bottom); (b) The UV-vis and PL spectra for P4 before the irradiation of LED and after irradiation in CH₂Cl₂ for 1 h; (c) The PL spectra for P4 before the irradiation of LED and after irradiation in Hexane for 5 h; (d) The PL spectra for P4 before the irradiation of LED and after irradiation in 1,4-dioxane for 5 h.

FIG. 9. Comparison of the color for different reaction mixtures. The comparison reaction mixtures of P1 and P4 before and after irradiation with blue LED for 6 h (a) in synthesis of 1a; (b) in synthesis of 2a, respectively. From FIG. 9a, pale-white is immediately formed for P4 when co-catalyst (ClCH₂CH₂Cl)₂NH₂Cl was added in reaction 1a, while the color of reaction mixture with P1 is still yellow. Meanwhile, for reaction 2a, both the reaction mixtures for P1 and P4 are yellow.

FIG. 10. The PL spectra for recycled CsPbBr₃ P1. The initial CsPbBr₃ NCs suspension in EtOAc (before mixing with any substrate, black line); CsPbBr₃ NCs suspension mixed with 1-benzylidene-2-phenylhydrazine, 2-bromoacetophenone and base before the irradiation of LED (red line); after the irradiation of LED for 12 h (blue line); the recycled CsPbBr₃ after centrifuging the reaction mixture and re-suspension in EtOAc (purple line); the recycled CsPbBr₃ applied for 2^{nd} time reaction (green line); the 2^{nd} time recycled CsPbBr₃ after centrifuging the reaction mixture and re-suspension in EtOAc (violet line). Inset: photograph of the recycled CsPbBr₃ (mixed with base residue) after EtOAc washing under UV light.

FIG. 11. PL spectra of P1 with addition of benzoic acid.

FIG. 12. PL spectra of P1 with addition of propionic acid.

FIG. 13. Stern-Volmer quenching studies of photocatalysts with air or organic substrate PhCOCH₂Br. (a) CsPbBr₃ NCs; (b) CdSe QDs; (c) Ir(ppy)₃ and (d) Ru(bpy)₃Cl₂.

FIG. 14. CsPbBr₃ NCs Emission quenching by 2-phenyl-1,2,3,4-tetrahydro-isoquinoline. (a) PL spectra of CsPbBr₃ NCs with the addition of 2-phenyl-1,2,3,4-tetrahydro-isoquinoline. (b) Stern-Volmer quenching study of 2-phenyl-1,2,3,4-tetrahydro-isoquinoline. k_{q}=1.8×10⁸ M⁻¹s⁻¹.

FIG. 15. CsPbBr₃ NCs Emission quenching by 4-phenylmorpholine. (a) PL spectra of CsPbBr₃ NCs with the addition of 4-phenylmorpholine. (b) Stern-Volmer quenching study of 4-phenylmorpholine. k_{q}=3.6×10⁸ M⁻¹s⁻¹.

FIG. 16. CsPbBr₃ NCs Emission quenching by benzylidene-malononitrile. (a) PL spectra of CsPbBr₃ NCs with the addition of benzylidene-malononitrile. (b) Stern-Volmer quenching study of benzylidene-malononitrile. k_{q}=3.4×10⁸ M⁻¹s⁻¹.

FIG. 17. CsPbBr₃ NCs Emission quenching by (E)-1-benzylidene-2-phenylhydrazine. (a) PL spectra of CsPbBr₃ NCs with the addition of (E)-1-benzylidene-2-phenylhydrazine. (b) Stern-Volmer quenching study of (E)-1-benzylidene-2-phenylhydrazine. k_{q}=8.8×10⁹ M⁻¹s⁻¹.

FIG. 18. CsPbBr₃ NCs Emission quenching by ethyl (E)-3-phenyl-3-(phenylamino)acrylate. (a) PL spectra of CsPbBr₃ NCs with the addition of ethyl (E)-3-phenyl-3-(phenylamino)acrylate. (b) Stern-Volmer quenching study of ethyl (E)-3-phenyl-3-(phenylamino)acrylate. k_{q}=4.9×10⁹ M⁻¹s⁻¹.

FIG. 19. CsPbBr₃ NCs Emission quenching by 2-bromoacetophenone in 1,4-dioxane. (a) PL spectra of CsPbBr₃ NCs with the addition of 2-bromoacetophenone in 1,4-dioxane. (b) Stern-Volmer quenching study of 2-bromoacetophenone in 1,4-dioxane. k_{q}=8.8×10⁸ M⁻¹s⁻¹.

FIG. 20. CsPbBr₃ NCs PL changing by adding 2,4′-dichloroacetophenone. No quenching was observed, indicating the initial ET transfer is difficult corroborating with the electrochemical driving force studies.

### DETAILED DESCRIPTION

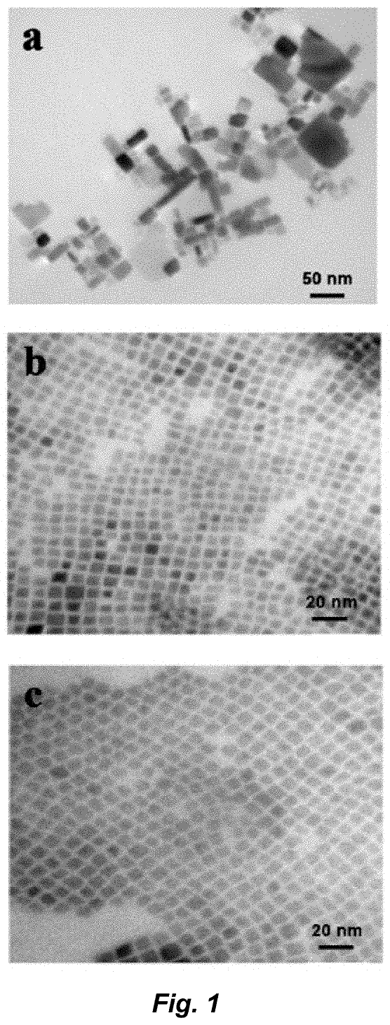

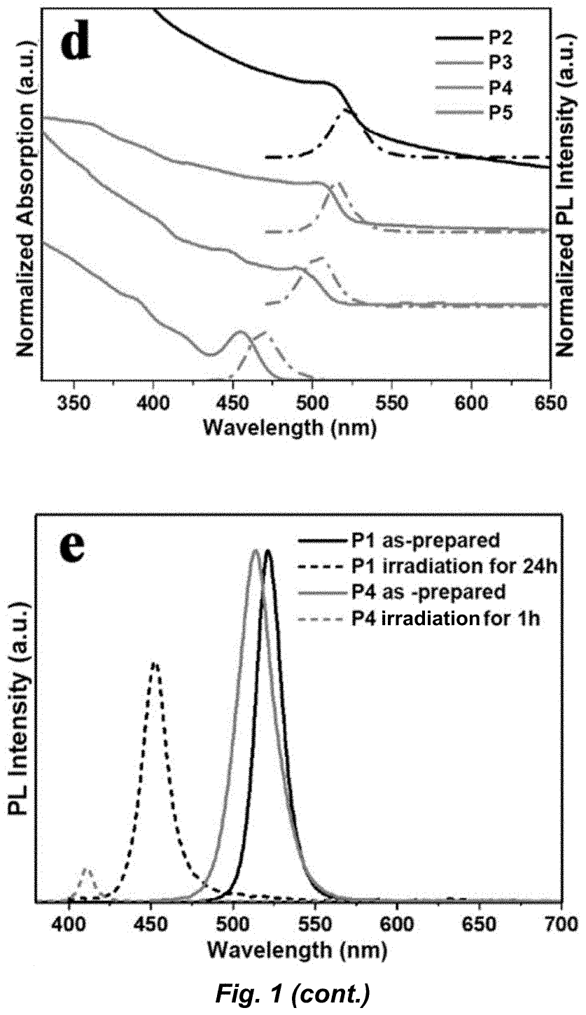

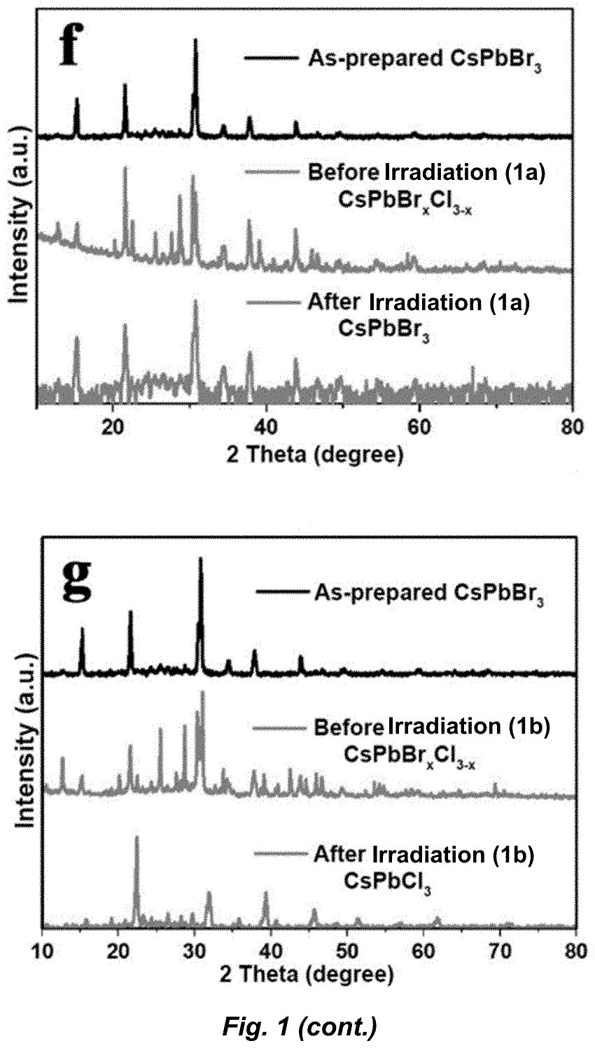

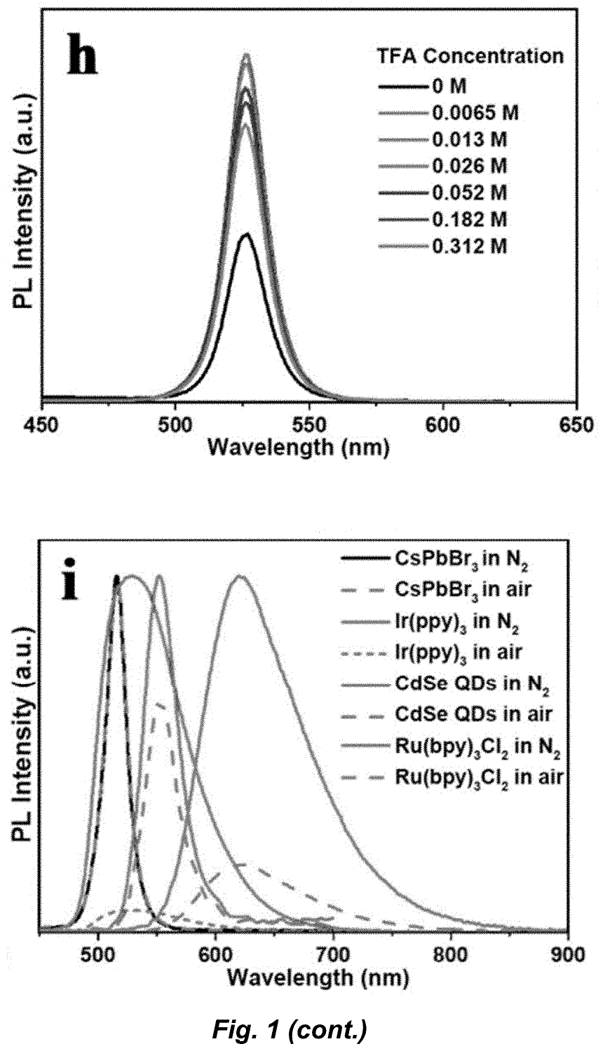

A general acceptance of perovskite nanocrystals for organic reactions has been demonstrated. C—C bond formations via C—H activation, C—N and C—O formations via N-heterocyclizations and aryl-esterifications can be achieved with moderate to high yields. Large size perovskites NCs with band energy determined by bulk CsPbX₃, in general provided higher yield for above reactions than perovskites quantum dots, probably due to a stability concern. A detailed stability study of perovskites regarding solvent type, ions, acidity has been explored. We also demonstrate that oxygen quenching of perovskite is less efficient. Therefore, perovskite colloids are much more active than most of those developed catalysts in air. Such tolerance may render perovskite a much broader activation for organic synthesis, particularly towards air. Mechanistic investigation further proves perovskites' excellent property towards photo-induced charge separation and transfer. Moreover, easy and wide bandedge tuning of the Pb-halide perovskites provides for achieving a key challenge in activating a broader range of organic substrates that require vastly different energy levels. Intentional or in-situ band-tuning experiment of CsPbBr₃ NCs exhibits that previously unachievable reactions, i.e. 2j, 3f, can be re-activate via a simple anion-exchange protocol (FIGS. 1 and 5). We envision that the photophysical knowledge that demonstrated in perovskite solar cell may be transformative for photocatalytic organic reactions. The broader application of this air-tolerant, cost-effective, easily-prepared, highly-active and band-tunable lead-halide perovskites may be of a revolutionary breakthrough in the photocatalysis of organic reactions.

Additional information and data supporting the invention can be found in the following publication by the inventors: Nat. Commun. 2019, 10, 2843 and its Supporting Information, which is incorporated herein by reference in its entity.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of or” “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below.

An alkylene is an alkyl group having two free valences at a carbon atom or two different carbon atoms of a carbon chain. Similarly, alkenylene and alkynylene are respectively an alkene and an alkyne having two free valences at two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered.

The term “aryl” refers to an aromatic hydrocarbon group derived from the removal of at least one hydrogen atom from a single carbon atom of a parent aromatic ring system.

The term “heteroaryl” refers to a monocyclic, bicyclic, or tricyclic ring system containing one, two, or three aromatic rings and containing at least one nitrogen, oxygen, or sulfur atom in an aromatic ring.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound.

A “solvent” as described herein can include water or an organic solvent. Examples of organic solvents include hydrocarbons such as toluene, xylene, hexane, and heptane; chlorinated solvents such as methylene chloride, chloroform, and dichloroethane; ethers such as diethyl ether, tetrahydrofuran, and dibutyl ether; ketones such as acetone and 2-butanone; esters such as ethyl acetate and butyl acetate; nitriles such as acetonitrile; alcohols such as methanol, ethanol, and tert-butanol; and aprotic polar solvents such as N,N-dimethylformamide (DMF), N,N-dimethylacetamide (DMA), and dimethyl sulfoxide (DMSO). Solvents may be used alone or two or more of them may be mixed for use to provide a “solvent system”.

The phrase “one or more” is readily understood by one of skill in the art, particularly when read in context of its usage. For example, one or more substituents on a phenyl ring refers to one to five, or one to up to four, for example if the phenyl ring is disubstituted.

### Embodiments of the Invention

This disclosure provides a method for photo-catalytic synthesis of an organic molecule comprising:
a) contacting a lead halide perovskite, first redox substrate, second redox substrate, and solvent to form a mixture, wherein:
   i) the first redox substrate comprises an alkylamine, and the second redox substrate comprises an alpha,beta-unsaturated carbonyl or alpha,beta-unsaturated nitrile, wherein a carbon-carbon (C—C) bond is formed via carbon-hydrogen activation; or
   ii) the first redox substrate comprises an imine or enamine, and the second redox substrate comprises an alpha-halocarbonyl, wherein a carbon-nitrogen (C—N) bond is formed via N-heterocyclization; or
   iii) the first redox substrate comprises an enamine or haloaryl, and the second redox substrate comprises an alpha-halocarbonyl or carboxylic acid, wherein a carbon-oxygen (C—O) bond is formed via cross-coupling; and
b) irradiating the mixture at a suitable wavelength to form at least one covalent bond between the first redox substrate and second redox substrate;
wherein the perovskite photo-catalyzes formation of the C—C, C—N, or C—O bond and the organic molecule is thereby synthesized.

In various embodiments, the perovskite is APbBr₃ wherein A is an alkali metal or N(C₁-C₆)₄. In some embodiments, the perovskite is CsPbBr₃. In some embodiments, the perovskite is in the form of a nanocrystal or colloid. In some embodiments, the perovskite has an average particle size of about 1 nanometer to about 150 nanometers. In various embodiments, the perovskite is pretreated with a trialkylsilylhalide.

In various embodiments, the perovskite is CsPbBr_{3-y}X_{y} wherein X is Cl or I, and y is 1-3. In some embodiments, the perovskite has an emission peak of about 450 nm to about 650 nm, about 590 nm to about 610 nm, about 550 nm to about 570 nm, about 490 nm to about 510 nm, about 450 nm to about 460 nm, about 400 nm to about 420 nm,

In some embodiments, the first or second redox substrate comprises an aldehyde, alpha-halocarbonyl, alpha, beta-unsaturated carbonyl or nitrile, alkylamine, imine, enamine, carboxylic acid, or haloaryl. In some embodiments, the alkylamine is an N-aryl substituted nitrogen heterocycloalkyl. In some embodiments, the enamine is an alkylidene hydrazine.

In some embodiments, the suitable wavelength is provided by a blue light emitting diode or compact fluorescent light (CFL) bulb. In some embodiments, the suitable wavelength is about 355 nanometers to about 465 nanometers.

In some embodiments, the solvent is dichloromethane, ethyl acetate, tetrahydrofuran, dioxane, hexanes, or toluene. In some embodiments, the mixture is contacting air, nitrogen, or oxygen. In some embodiments, the mixture is contacting an acidic or basic additive. In some embodiments the acidic additive is an inorganic acid or organic acid. In some embodiments the basic additive is an inorganic base or organic base. In some embodiments, the mixture is contacting a co-catalyst.

In various embodiments, the perovskite photo-catalyzes formation of the C—C bond. In various embodiments, the mixture is contacting oxygen. In various embodiments, the mixture is contacting an acidic additive.

In various embodiments, the perovskite photo-catalyzes formation of the C—N bond. In various embodiments, the mixture is contacting a basic additive and/or air.

In various embodiments, the perovskite photo-catalyzes formation of the C—O bond. In various embodiments, the mixture is contacting a transition metal co-catalyst and/or a basic additive. In various embodiments, the co-catalyst is a nickel catalyst.

In some embodiments, the co-catalyst is a nickel catalyst or an alkyl ammonium halide catalyst. In some embodiments, the at least one covalent bond formed is a C—C bond, C—O bond, C—N bond, or combination thereof. In some embodiments, the at least one covalent forms a heterocycle.

### Results and Discussion

General acceptance of perovskites for organic synthesis. Perovskite colloidal suspension (CsPbX₃: E_{CB}=−1.2˜−1.4 V, E_{VB}=+0.6˜+1.5 V, all vs SCE; CB: conduction band; VB: valence band) are effective catalysts for several fundamental organic reactions under visible light as shown in Scheme 1. Direct C—C bond formations are observed via C—H activation of aldehydes (1a, 1b) or tertiary amines (1c, 1d). The scope of the former reaction is not only limited on previously explored C—Br weaker bonds, but also covers stronger C—Cl bond. The absence or presence of oxygen is the key to lead to chain-extension product (1c) or an unexpected cyclization reaction (1d). C—N bond formations via direct N-heterocyclizations forming pyrazoles (2a-f) and pyrroles (2g-i), critical reaction for pharmaceutical development, are realized in high yield with perovskite at room temperature. C—O bond formation via aryl-esterification (3a-f) was achieved with a Ni co-catalyst. The respective reaction conditions are also optimized with regards to solvents, types of perovskites, air-tolerance, co-catalysts, and reaction time etc. (see Table 2-9 for details). Catalyst loading has also been explored (Table 2-8) and respective minimum loading for typical reactions of ˜0.1-0.5 mmol has been listed in Scheme 1. These reactions result in respective products in moderate to high yields without need for anaerobic sparging. The scopes of each aforementioned reaction were explored with various functional groups. (Scheme 1 and Methods for details) As expected, control experiments reveal no product in the absence of photocatalyst or light.

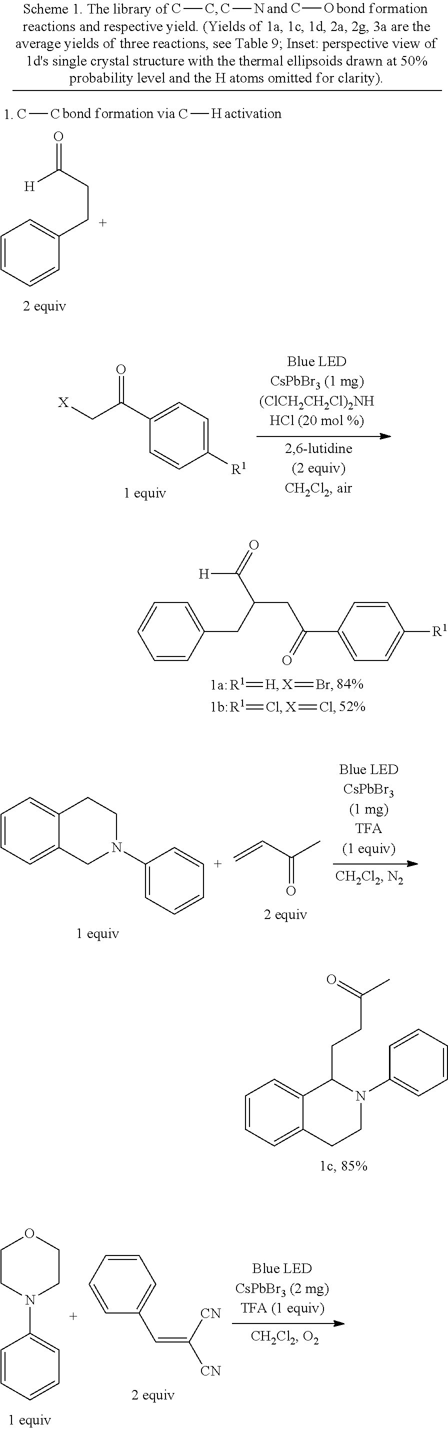

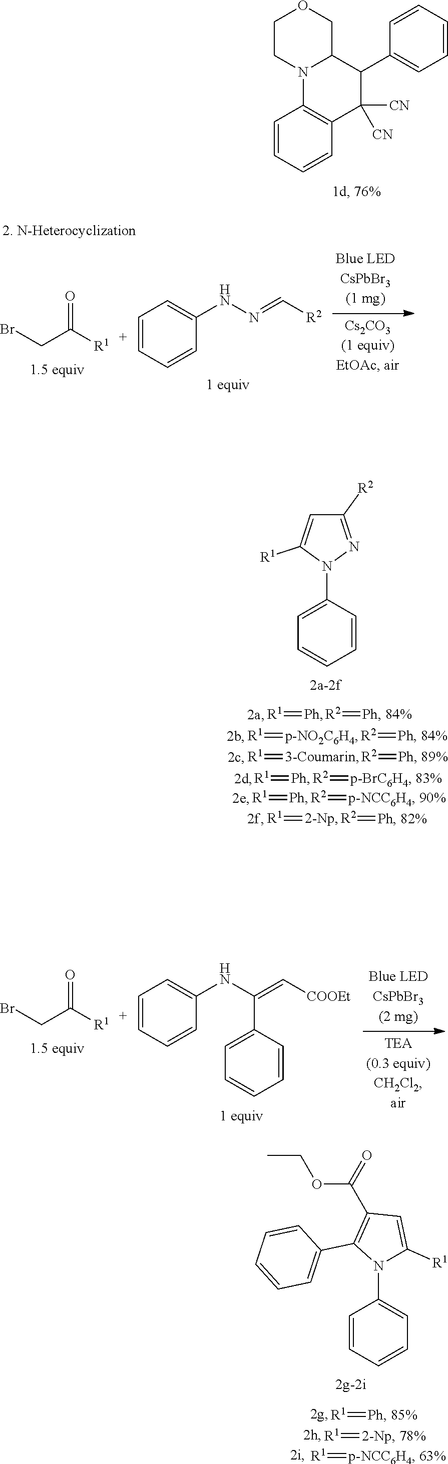

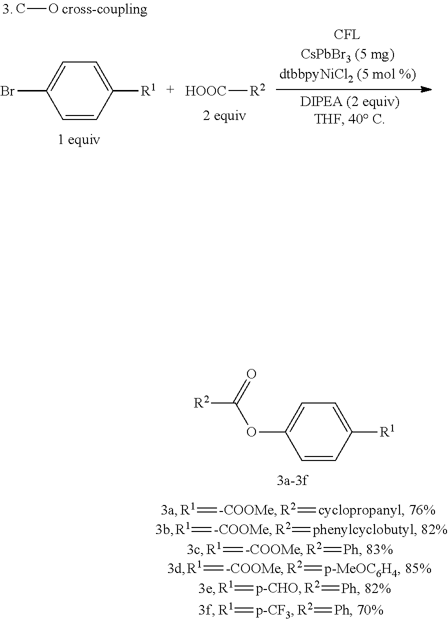

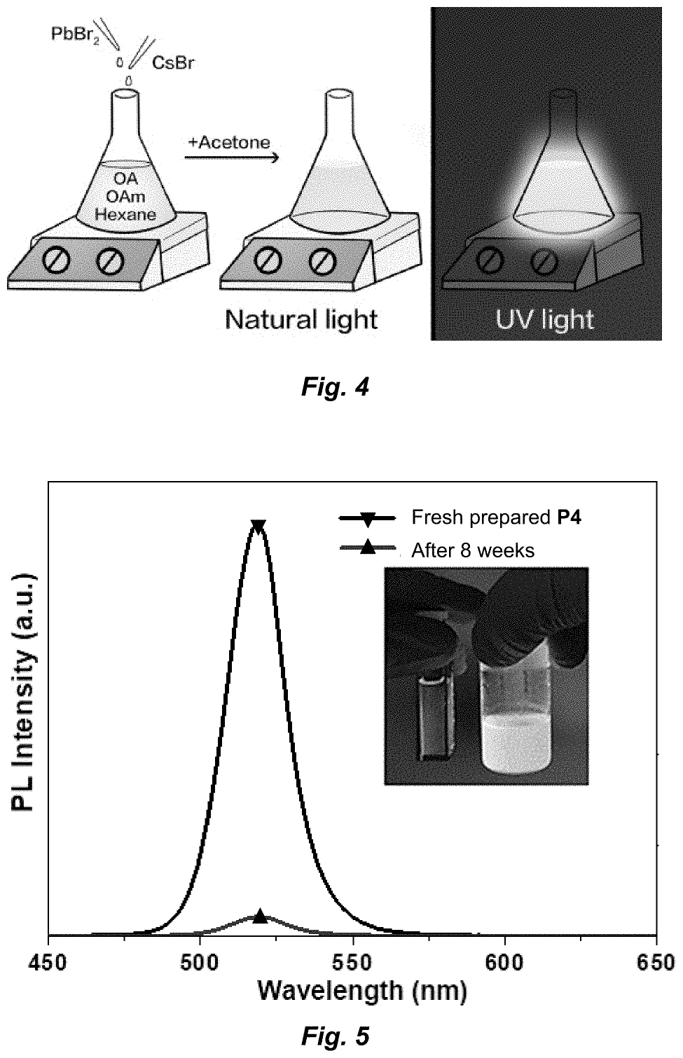

Perovskite's size effect. The perovskite colloids, P1, described above are readily synthesized according to a previous report (J. Am. Chem. Soc. 2019, 141, 733) via directly mixing of readily available low-cost starting materials, PbX₂ with CsX, in an open vial under bench-top conditions (FIG. 4). The resulting gram-scale emissive perovskite colloids exhibit a broad size-distribution, ca. 2˜100 nm (FIG. 1a). The observation together indicates a bandgap energy of 2.4 eV that well matches the bulk CsPbBr₃ bandgap. The synthesized colloids are too large to be in the quantum-confinement regime (FIG. 1a). Thus, for the system we are considering most colloids within the ensemble are larger than the Bohr radius, and hence the bandedges are determined by bulk bandedges and quantum-confinement effects do not contribute.

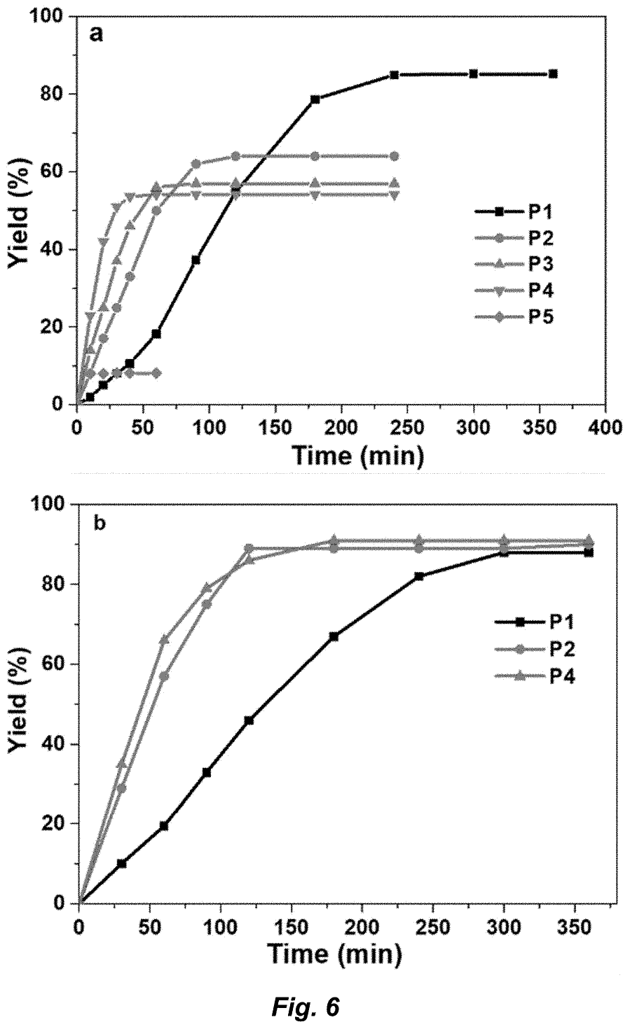

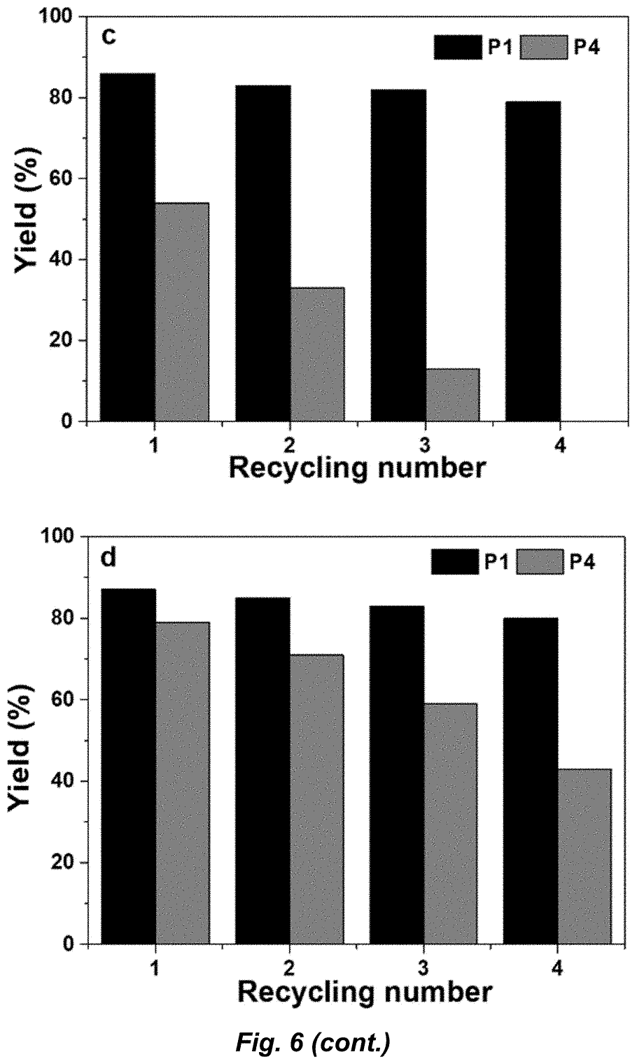

In contrast, using a high temperature synthetic method, we also synthesized size-controlled CsPbBr₃ NCs (P2 14 nm, λ_{PL}=521 nm; P3, 9 nm, λ_{PL}, =515 nm; P4, 6 nm, λ_{PL}=508 nm; P5, 4 nm, λ_{PL}=467 nm, FIG. 1b-d and FIG. 5). As shown in FIG. 1d, these NCs show a blue-shift probably due to quantum confinements. The photocatalytic ability has also been explored in the same reaction condition. In C—H activation, at the early stage of the reaction, we find that smaller size NCs, i.e. P2-P4 show a higher initial reaction rate compared to the original synthesized P1 NCs. (FIG. 6). However, small size NCs' catalytic reactivity diminished quickly. When breaking a C—Br bond to form 1a, the reaction yield is recorded as 54-64% using P2-P4 in less than 40 min, and longer reaction time leads to a marginal increase of the yield of 1a. Much lower yield, ˜8% was observed within P5 probably due to a significant blue-shift leading to less visible absorption. Whereas using P1, the reaction rate is slower, however, the yield continuously increases and reaches 85% in ca. 5 hours.

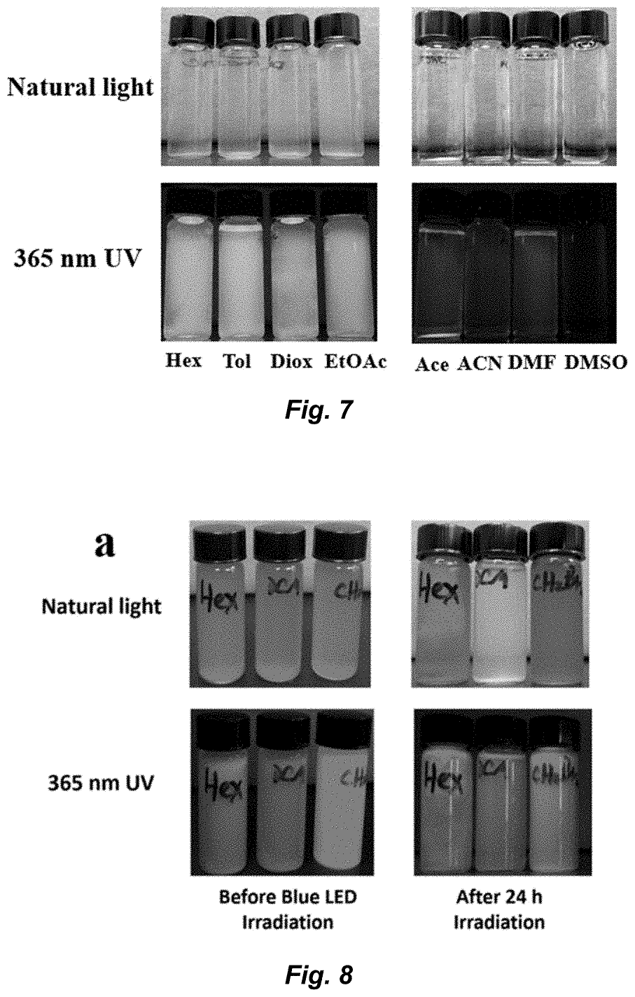

We suspect that small size NCs have higher surface area-to-volume ratio (Table 10), hence a faster rate at the early stage. However, detrimental effects, i.e. moisture residue in solvent are inevitable. Such effects are more prominent on small size NCs than P1. We assume if the desired photocatalysis is slower than perovskite decomposition, the reaction yield may be of significant discrepancy between small and large size NCs. Such assumption is corroborated with reaction 1a described above. In contrast, if the decomposition is not prominent, the yield discrepancy is less obvious. In fact, in 2a, perovskite is stable in a pre-dried non-halide solvent ethyl acetate (FIG. 7). 2a is produced in 86% yield with P2 in 2 hours, 87% using P1 in 6 hours (FIG. 6). Overall, small size NCs in general promote a faster reaction rate, but not necessarily a higher yield unless presenting in a perovskite friendly reaction environment. Considering synthesis merits, large size NCs in general provide higher yield although a longer reaction time in a scale of 6 hours or higher is required.

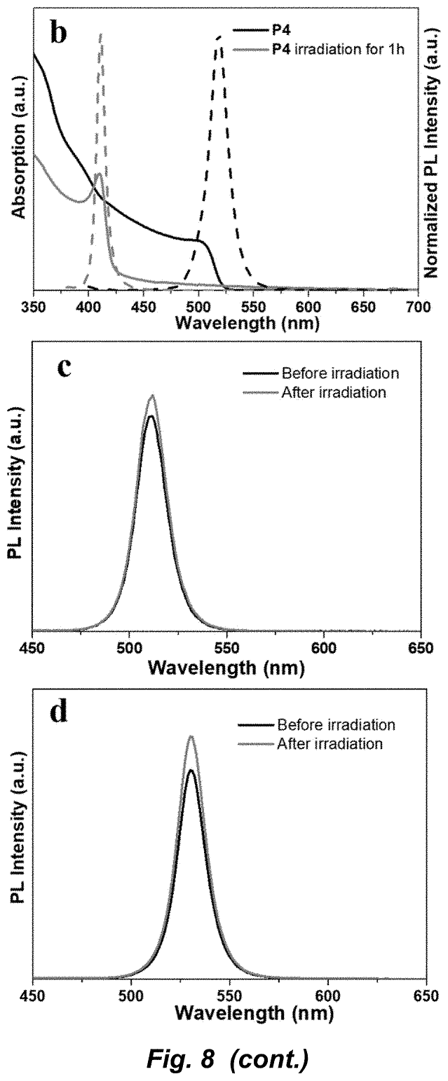

Stability and reaction condition tolerance. Pb-halide perovskites' photovoltaic performance perishes over moisture, impeding the wide commercial application of such materials as solar cells. The stability is quite distinct if perovskites are to be applied to organic synthesis in which more critical parameters may influence the stability of perovskites, i.e. solvent type, ions, acidity, etc., and further manipulate the catalytic ability. Thus, these parameters are evaluated individually for a better understanding of perovskite photocatalysis. A quite strong stability of P1 in organic solvents was indicated by no obvious PL changes of CsPbBr₃ for several weeks in less polar organic solvents. (Note that P1 is not stable in polar solvents, i.e. acetone, acetonitrile, DMF, DMSO, FIG. 7). However, P2-P5 are less prominent and significant PL diminishing is observed. (FIG. 5) Interestingly, under the irradiation of LED, PL blue-shift of P1 in CH₂Cl₂ are observed in 24 hours. (FIG. 1e) Such changes are significantly magnified on P2-P5 as shown in FIG. 1e and FIG. 8, absorption and PL blue-shift within in 1 hour, whereas no obvious PL changes are observed in non-halide solvents. This observation may be attributed to a photoinduced fast halide exchange for CsPbBr₃ with CH₂Cl₂ as previously reported (Catal. Sci. Technol. 2018, 8, 4257).

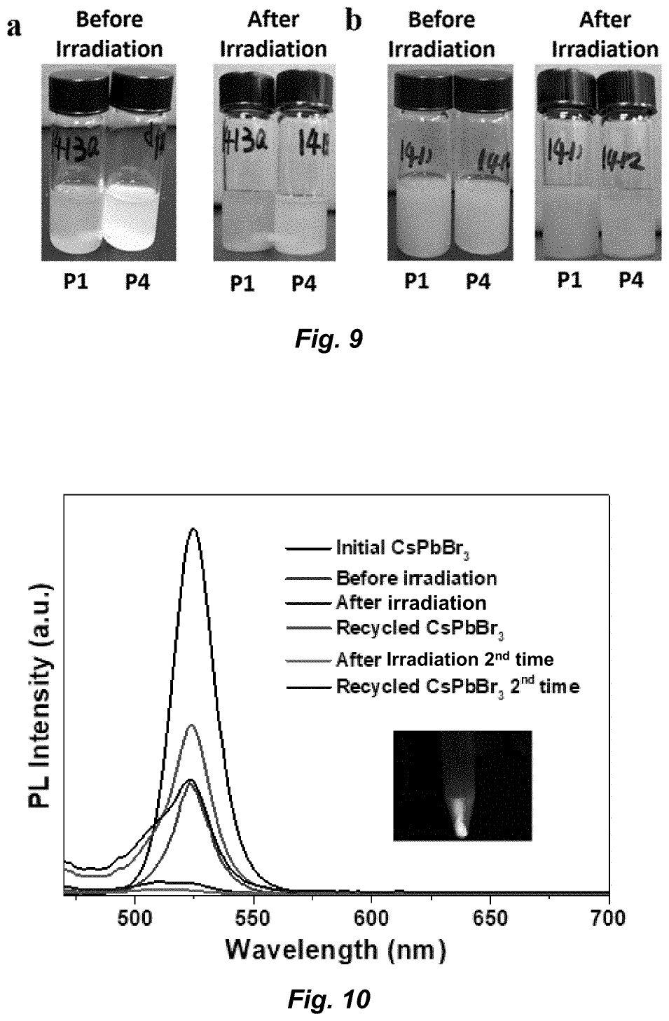

Next, we evaluate the ion effect in perovskites' photocatalysis. Perovskite is to be sensitive to both inorganic cations and anions. In our photocatalytic setup, co-catalyst (ClCH₂CH₂Cl)₂NH₂Cl in reaction 1a, leads to an initial PL blue-shifted due to anion-exchange forming CsPbBrₓCl₃₋ₓ, confirmed by XRD (FIG. 1f). It is interesting to point out that co-formation of Br ion during reaction 1a, may further exchange with the CsPbBrₓCl₃₋ₓ and stabilize the perovskite NCs. Such stabilization is evidenced by the after-reaction catalyst characterization in which XRD indicates that the isolated photocatalyst solid was corresponding to CsPbBr₃ and surprisingly, no peak has been assigned to CsPbCl₃ (FIG. 1f). This is probably because the co-formation Br ions are in chemical equivalency and its concentration is significantly higher than that of Cl. Therefore, a Br compensated and stabilized CsPbBr₃ P1 photocatalyst system is thus observed. (FIG. 9) In contrast, reaction 1b employing Cl-substrates leads to a fully-exchanged CsPbCl₃ after reaction (FIG. 1g). Overall, perovskite P1 shows a much better stability during the reaction 1a, in which the NCs can be isolated from the previous reaction mixture via centrifuging and then re-suspended for a new reaction under identical conditions for at least four cycles with slightly PL blue-shift, whereas small NCs P4's recycling ability is limited

(FIG. 6). As comparison, when free halide anions are absent, for example in reaction 2a in EtOAc solution, the overall stabilities for P1 and P4 are enhanced and result in an improved recyclability in such perovskite friendly environment (FIG. 9-10).

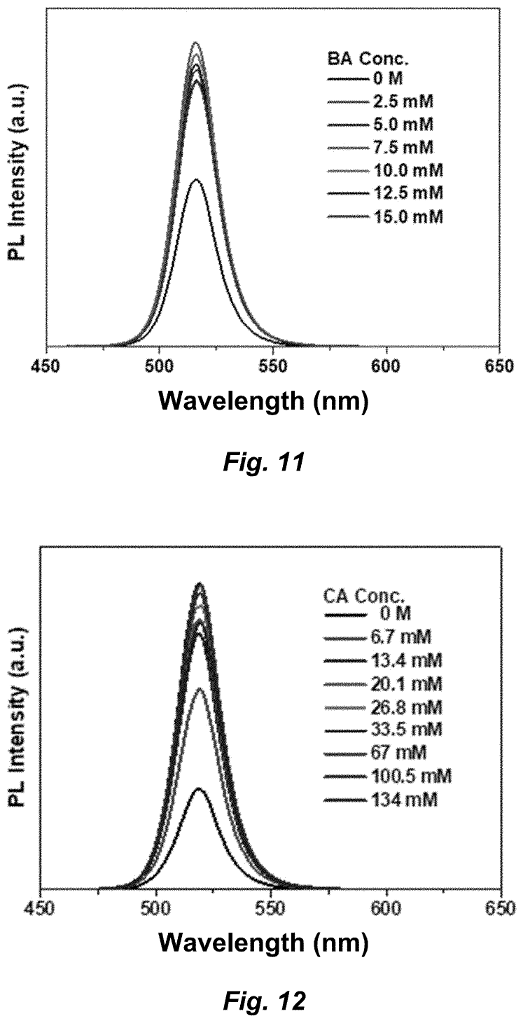

Acidity or free protons in perovskite reaction mixture may play a role in organic synthesis. For instance, carboxylic acids such as propionic acid, benzoic acid or trifluoroacetic acid (TFA), were employed as the co-catalyst (1c and 1d) or as a substrate (3a-3f). Thus, we first measured the PL for perovskite NCs with different acids to elucidate the tolerance of acidic conditions. Interestingly, as shown in FIG. 1h and FIG. 11-12, a PL enhancement of P1 was observed upon the addition of benzoic acid, propionic acid, and also TFA. This is corroborated with previously observed PL enhancement using thiophenol, phosphoric acid etc. The PL enhancements are probably because carboxylic acid function as the capping ligand by the strong hydrogen bonding with surface halide ions and may also account from a strong interaction between carboxylic acid and Pb atoms. Acid binding with defects on perovskite may also lead to an enhanced PL performance. The maximum PL was observed using TFA at a concentration of ca. 6.5-13 mM, more acid leads to a diminishing PL probably because large number of protons may start to initiate a deactivation process. Interestingly, such optimized TFA concentration also leads to a maximum product yield of 1c and 1d as shown in Table 4-5, indicating that a high PL of the photocatalyst may increase the catalytic conversion. Therefore, non-halide organic acid may not only stabilize the perovskite NCs, but also may increase the overall catalytic efficiency for respective reactions.

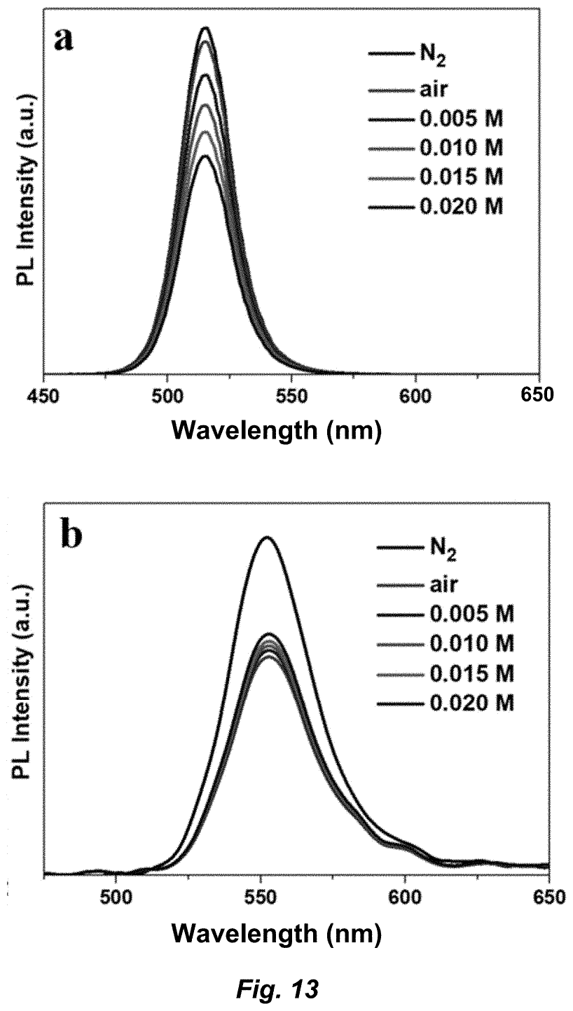

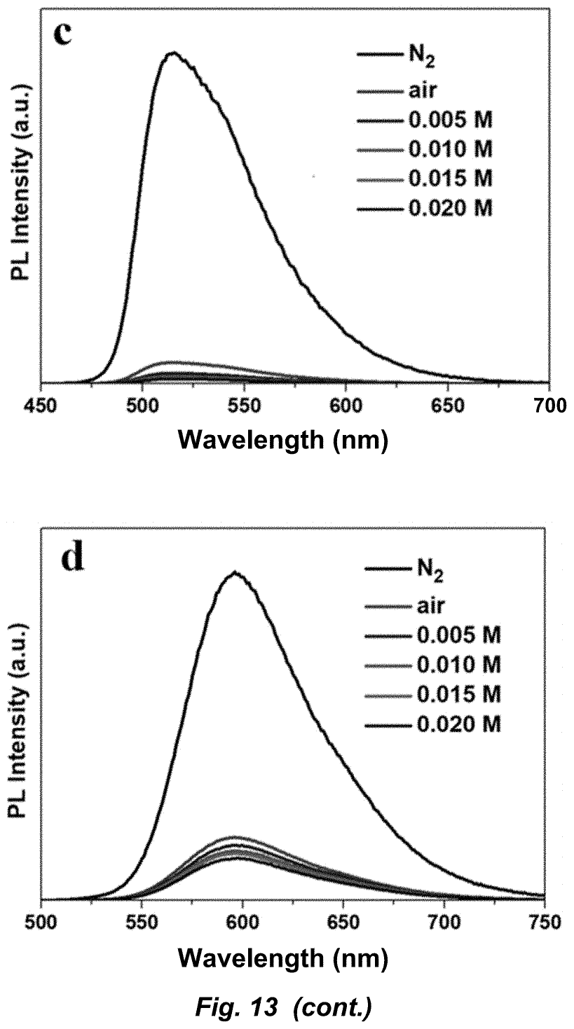

Key catalytic parameter comparison with other photocatalyst. Air-tolerance is important for the practical end-use of chemical synthesis. One distinct advantage of our colloidal system is that the organic reactions observed here occur without the need for N₂-sparging. In stark contrast, molecular photocatalyst necessitates air-free reaction conditions. The key difference here is that the perovskite NCs likely undergo faster quenching from the organic substrates, while quenching from air is negligible. (FIG. 1i and FIG. 13) The reverse is true for most cases of molecular catalysts—i.e., 02 quenching is substantial and competitive with the catalytic reactions, leading to poor catalytic results. Hence, yields of reaction 1, 2, and 3 in air with perovskite are significantly higher than with others. (Table 1-8) For instance, 1a were obtained in 85% yield in air using perovskite, but only resulted in trace amount with Ru(bpy)₃²⁺. These results suggested that perovskite may exhibit a broad tolerance, particularly towards air.

TABLE 1
Comparison of photocatalysts for corresponding reactions in air or in oxygen.

| | Yield (%) ^{b} | | | | | TON (based on CsPbBr₃) | | | | |
|---|---|---|---|---|---|---|---|---|---|---|
| Photocatalyst ^{a} | 1a | 1c | 1d | 2a | 3f | 1a | 1c | 1d | 2a | 3f |
| CsPbBr₃ P1 | 84 | 85 | 76 | 84 | 70 | 9,100 | 830 | 280 | 380 | 33 |
| Ru(bpy)₃(PF₆)₂ | Trace | 60 | 25 | Trace | N.R. | — | 60 | 25 | — | — |
| Ir(ppy)₃ | 79 | N.R. | N.R. | 63 | 65 | 79 | — | — | 63 | 33 |
| CdSe QDs (525 nm) | Trace | Trace | N.R. | N.R. | N.R. | — | — | — | — | — |

^{a} bpy = 2,2′-bipyridine;
ppy = ortho-metalated 2-phenylpyridine;
^{b} average yield using for P1.

Catalytic turnover number (TON) is compared and listed in Table 1. Heterogeneous catalyst, i.e. 3.0 nm CdSe QDs were reported to optimally render a TON of 79,100 (based on QD's molecular weight Mw, 88,000 g mol⁻¹) in glove box. However, in our condition under air, no yield (nor TON) of 1, 2 and 3 can be obtained using CdSe QDs. In addition to air-sensitivity, CdSe's performance was also dependent on size and capping ligands. While changing capping ligand on perovskite plays little role in the yield as shown in Table 3-5. This is probably because the capping ligands (e.g., n-octylammonium) that stabilize perovskite colloids function as A site to the perovskite APbX₃ structure, hence no extra stabilization protocol is required using perovskite nanocrystal for photocatalysis. Using the method in CdSe QDs to calculate TON, P2 NCs (14 nm, based on Mw, 8,015,000 g mol⁻¹, P1-P5 TON see Table 10) renders 2,565,000. Perovskites' heterogeneous catalytic ability is validated via regaining strong PL after recovering the catalyst via centrifuge after reaction (FIG. 10). To compare TON with molecular catalysts, TON calculation based on mole of metal (independent of size, CsPbBr₃, 579.8 g mol⁻¹) was carried out instead. For instance, four cycles of the reactions afford a TON, at least 9,100 for 1a (Table 1). Overall, one or two orders of higher TONs under our condition are observed using perovskite than others, except reaction 3 in which TON may rely on both perovskite and Ni co-catalyst.

Higher activity of perovskite than other photocatalysts may account from the intrinsic photophysical properties on charge separation and transfer. For example, the perovskite NC's ultrafast interfacial electron and hole transfer dynamics has been observed. First, negligible electron or hole trapping has been found in perovskite NCs, facilitating photoredox catalytic cycle. In the presence of organic substrates (as electron or hole acceptors in photoredox organic synthesis), photon-induced excitons in perovskite can be efficiently dissociated and separated. For instance the half-lives of electron transfer to an organic electron-acceptor is reported to be ˜65 ps, while charge recombination rate is reported about ˜2 orders slower. The hole transfer dynamics from perovskite to an organic substrate is also reported to be 20 times faster than its recombination. Such observation is also corroborating with our previously reports on the ultra-slow recombination velocity of perovskite both in CsPbBr₃ and CSPbI₃ single crystals and films. Overall, the lack of electron and hole traps and fast interfacial electron transfer and hole transfer rates are imperative that may enable highly efficient perovskite induced photocatalysis. In fact, the superior performance is not surprising given that when employed in photovoltaics, the Pb-halide perovskites also perform much better (PCE, 24.2%) compared to transition metal-based dye-sensitized solar cells (11%), QD photovoltaics (12%) and organic photovoltaics (12%).

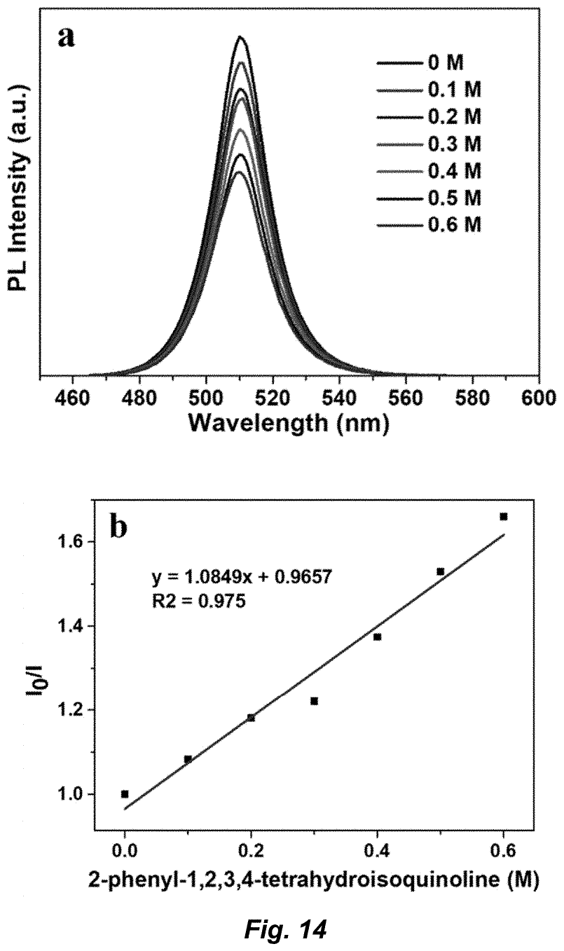

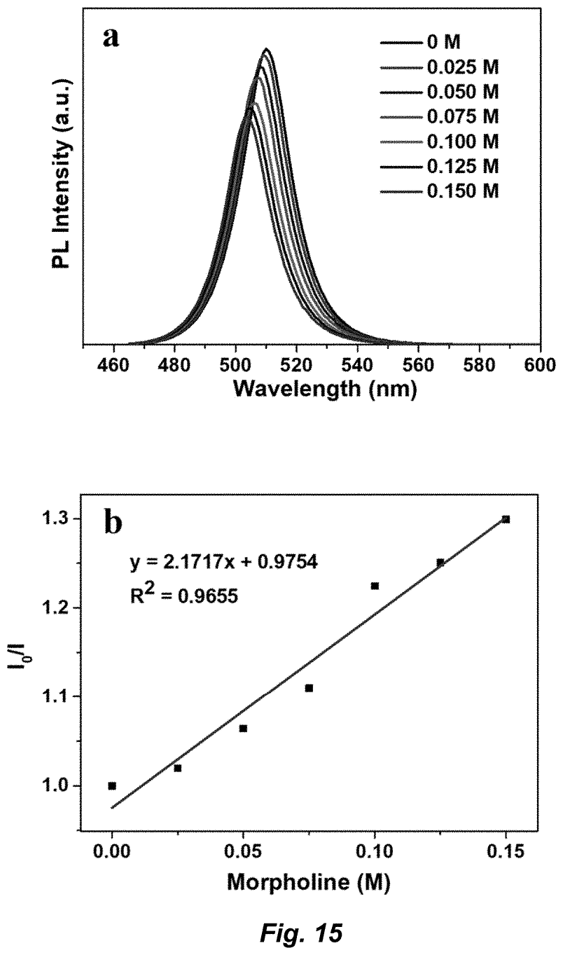

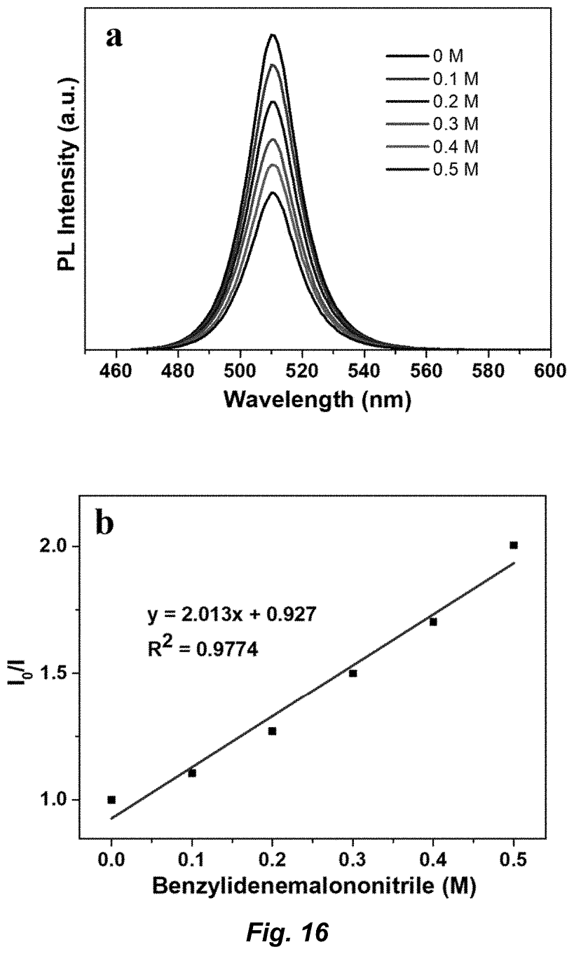

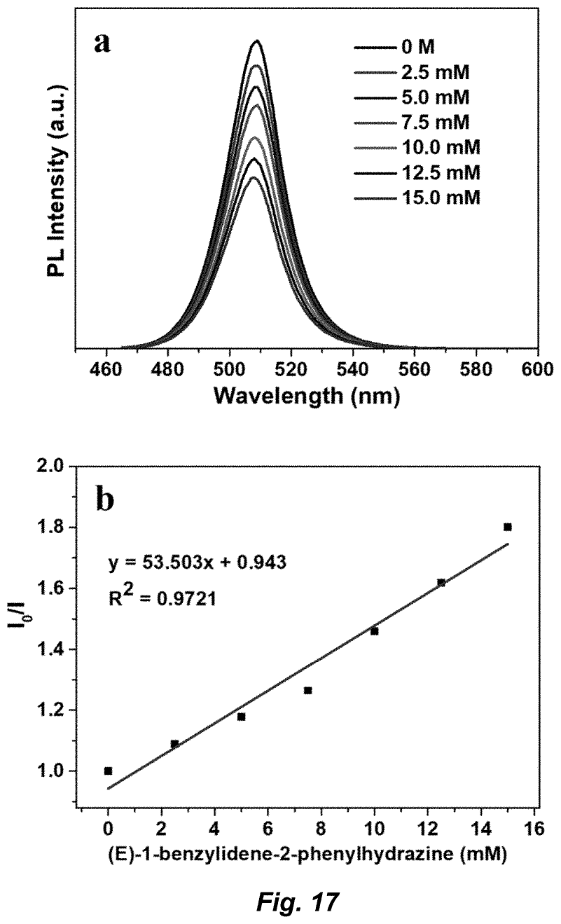

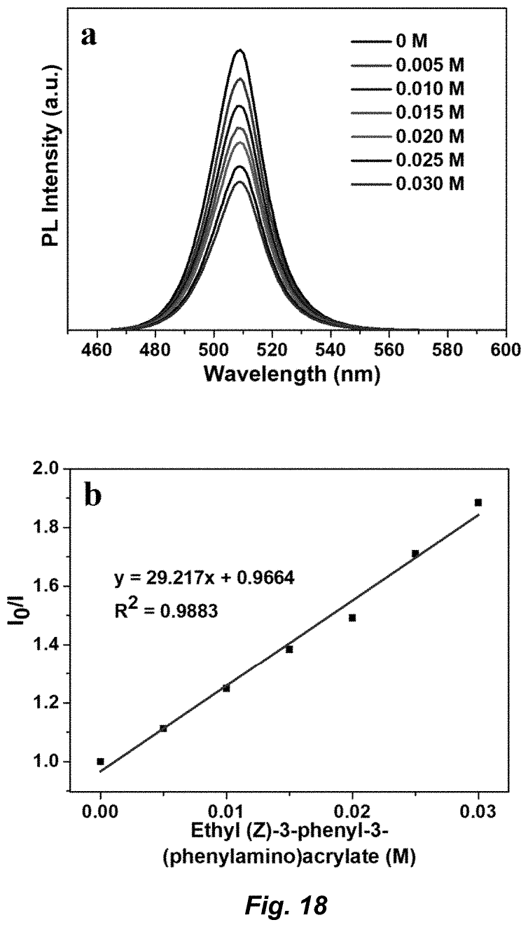

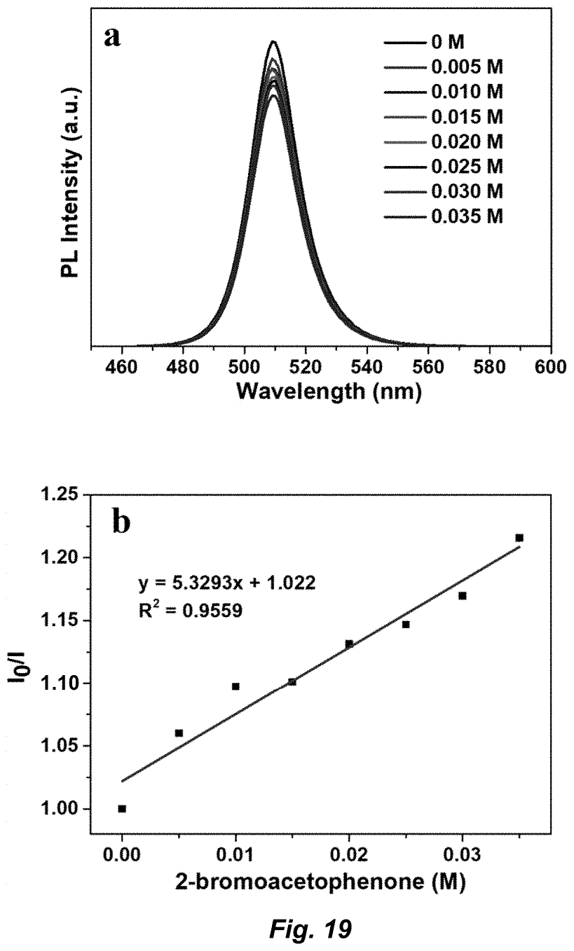

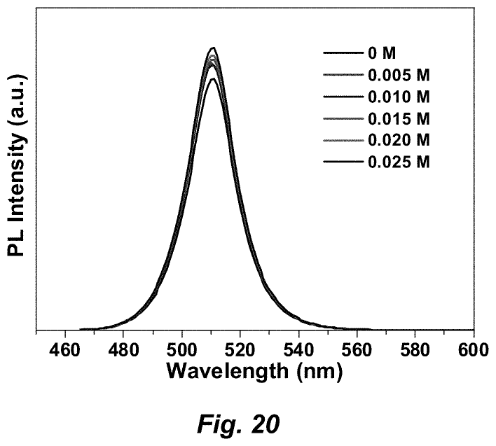

Mechanism. Oxygen may be of an essential component in certain photoredox reactions. For instance, in Scheme 2a, radical addition product 1c is achieved in nitrogen atmosphere while in a similar setup, air or oxygen atmosphere produces a ring-closure 1d (crystal structure provided in Scheme 1). Oxygen is found to be the key reagent as the hydrogen atom acceptor that further induced the C—H activation on phenyl rings. As shown in Scheme 2, the reaction mechanisms are proposed in which the key radical intermediates have been investigated. Upon Stern-Volmer studies (FIG. 14-20), perovskite PL quenching by 1d-A was observed (k_{q}=3.6×10⁸ M⁻¹s⁻¹, details see FIG. 15) and resulted in 1d-B radical in the presence of oxygen. Intermediate 1d-B and 1d-C have been verified via radical trapping experiment employing 2,2,6,6-tetramethyl-1-piperidinyloxy (TEMPO) as a radical scavenger, through LC-MS. In the absence of oxygen, radical 1c-B is also confirmed by TEMPO-trapped product and further verified by the self-coupling 1c-C via ¹H NMR. It is worth mentioning that the presence of air leads to more 1c-C formation and ultimately diminishes the yield of 1c.

Scheme 2b shows the proposed mechanism of C—N formations, in which both oxidative (ET, 2a-A) and reductive quenching product (HT, 2a-B) in reaction 2a have been trapped by TEMPO (either observed via ¹H NMR or LCMS), indicating a strong charge separation and transfer ability induced by perovskite. This pathway is similar to our previous mechanism exploration in α-alkylation of aldehydes. Radical coupling between 2a-A and 2a-C leads to the intermediate of 2a-D. Then C—N formation via intramolecular cyclization and a final dehydration leads to the pyrazole product 2a. In contrast, the radical formation from 2g-B via direct HT has not been observed, instead 2g-C was verified via radical-trapping, likely demonstrating a different mechanism of pyrrole formation as shown in Scheme 2b.

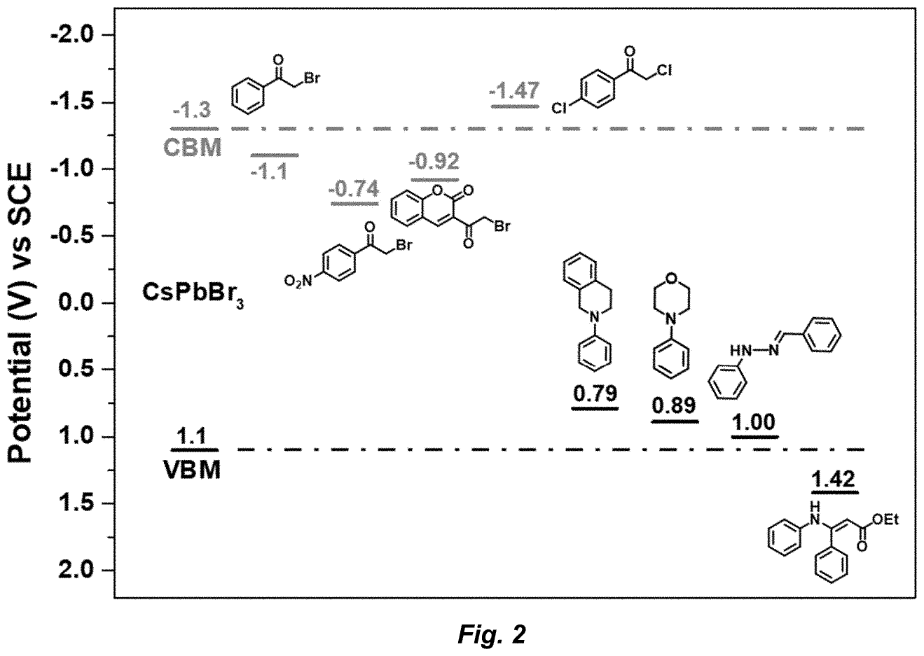

To further elucidate the reaction mechanism, electrochemical studies were conducted. According to the comparison between redox potentials of the key substrates and the band energy of perovskite, the respective driving force is listed in FIG. 2. Driving force for HT in reaction 1c, 1d and 2a is observed among ˜0.1 to 0.3 eV, consistent with the Stern-Volmer quenching results (FIG. 14-20) as well as the mechanistically verified intermediates in Scheme 2. However, 2g-B disfavors HT due to a more positive oxidation potential (Eₒₓ, 1.42V vs SCE), corroborating with the previous observation that direct radical forming from 2g-B is difficult, unlike reaction 2a pathway. Moreover, driving force for ET is also listed from ˜0.2 to 0.5 eV, confirming our discussion on ET in Scheme 2. However, noticeable exception, 2,4′-dichloroacetophenone, though presenting a more negative reduction potential (Erect, −1.47V vs SCE), still reacts to form respective pyrrole. We postulate that in-situ band-tuning of perovskite may play a role here and is discussed below.

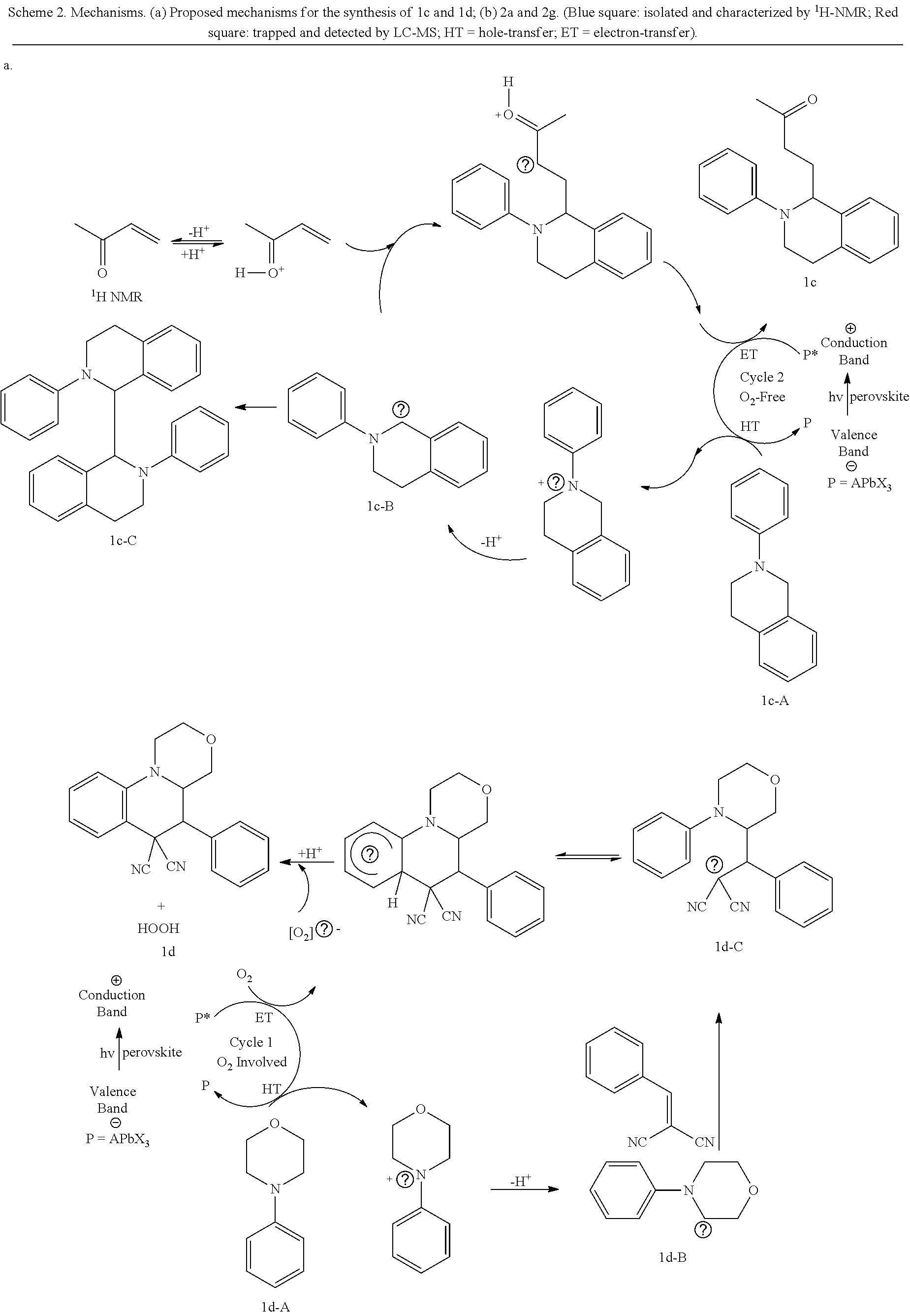

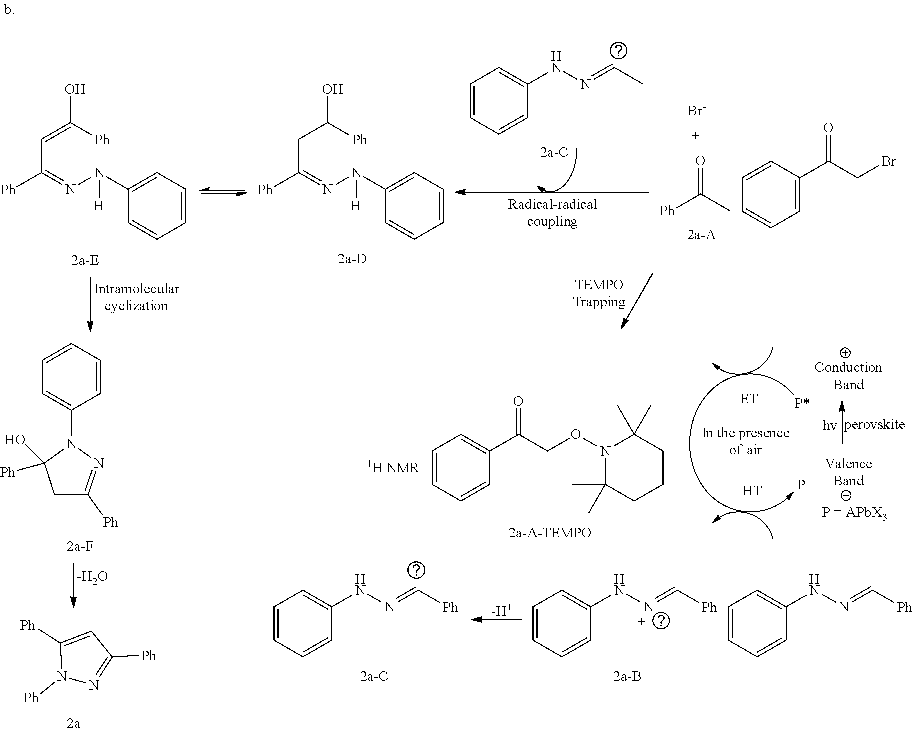

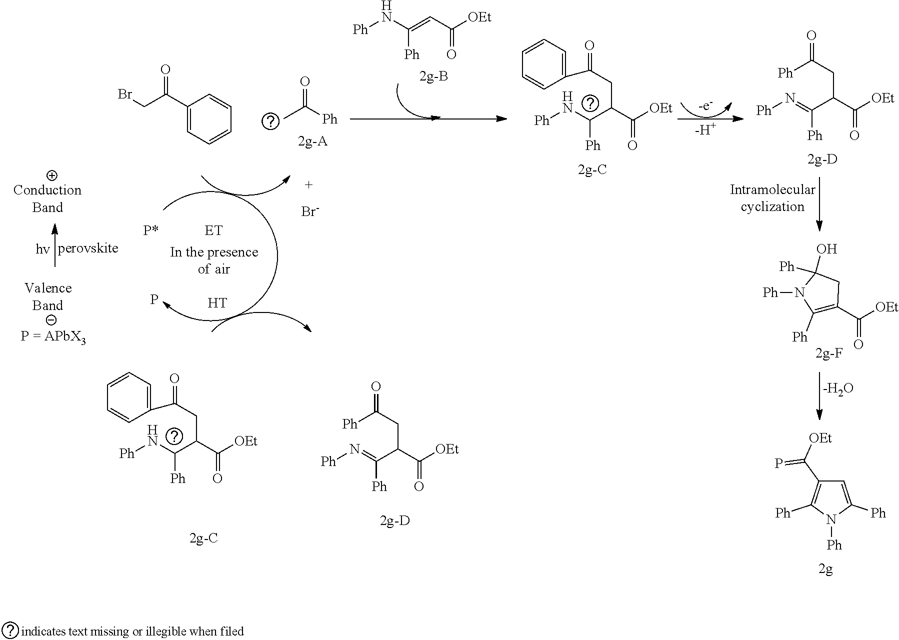

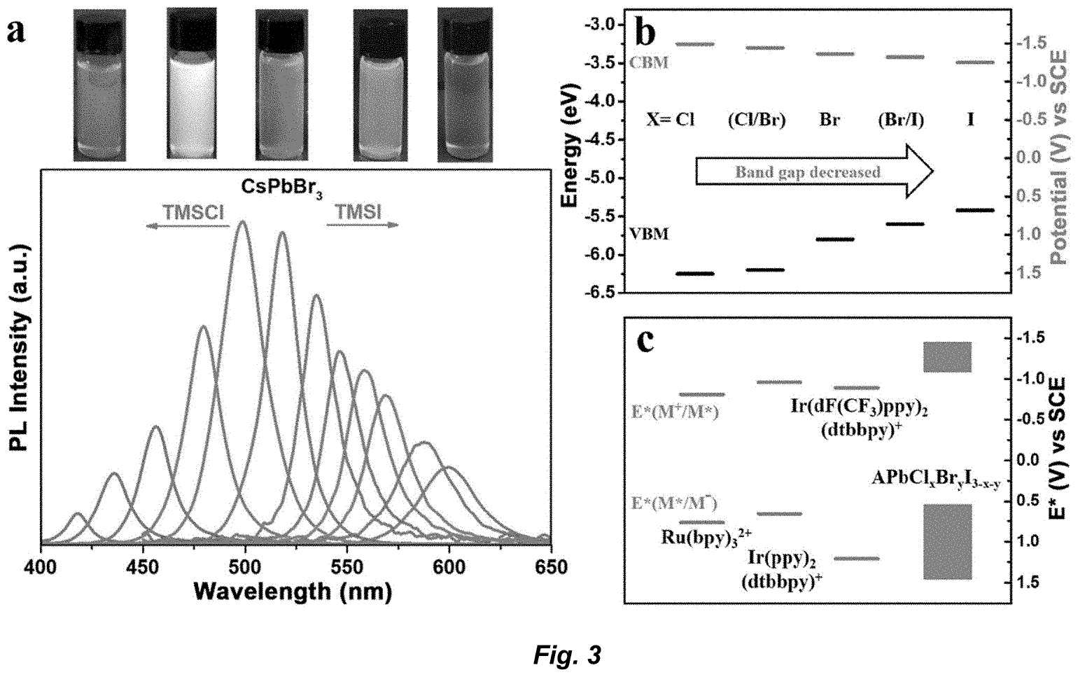

Unique band-tuning of perovskite. As discussed above, the perovskite NCs P1 are too large to be in the quantum-confinement regime and the majority of the NCs within the ensemble are larger than the Bohr radius. Thus, the band energy of our photocatalyst, analogues to excited state redox potentials, E* in molecular catalyst, is determined by the bulk bandedges. Bandedge-tuning is achievable by simply mixing of different ratio of halides. We also observed that in-situ ion exchange using P1 results in band-tuning (FIG. 3a). In theory, as shown in FIG. 3b-c the bandedges of perovskite after tuning covers most of the E* of the known Ru or Ir molecular photocatalysts.

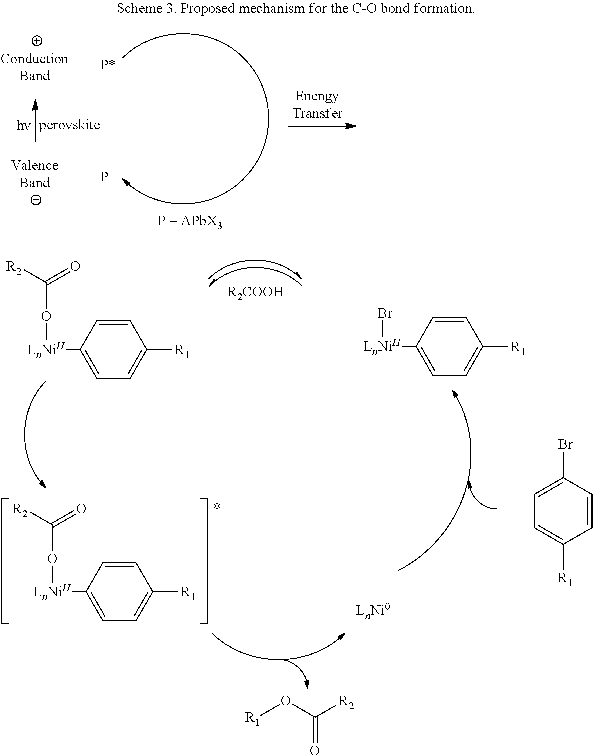

The band-tuning is of critical importance for a photocatalyst to activate different types of substrates. For example, C—O formation reaction 3 is also proposed and shown in Scheme 3 similar to previous reported mechanism (Science 2017, 355, 380). It is reported that energy transfer from triplet excited state of Ir photocatalyst is the key for Ni complex activation thus resulting in an efficient reductive elimination for C—O bond formation. Triplet energy (E_{T}) exploration from Ir(ppy)₃ derivatives via modifying the substitution group on ppy ligand demonstrated that a higher correlation between E_{T} and the production yield. Specifically, a higher E_{T} results in a higher yield. As shown in Scheme 4, in our perovskite system, 3f is produced in trace amount if CsPbBr₃ is employed with dtbbpyNiBr₂ co-catalyst, comparing to 78% with dtbbpyNiCl₂. While in Ir photocatalysis, the different halides on Ni co-catalyst only play a marginal effect. We suspect that an in-situ ion-exchange from NiCl₂ may result in a blue shift of perovskite, similar to the increasing E_{T} in Ir system, thus leading to a significantly higher yield of 3f using co-catalyst dtbbpyNiCl₂. To further confirm such hypothesis, we have conducted a systematic band-tuning experiment to demonstrate the correlation between the bandedges and the yield of 3f. In a typical experiment, perovskite CsPbBr₃ is employed with NiBr₂ co-catalyst, but tuned using a reported agent, i.e. trimethylsilyl chloride (TMSCl). We find that shifting the bandgap to higher values, by mixing with chloride to form CsPbClₓBr₃₋ₓ, increases the yield of 3f, similar to elevate E_{T} in Ir system. However, more Cl component is not always beneficial for this type of reaction. As shown in FIG. 5a, PL intensity is significantly lower when Cl is incorporated into perovskite. Higher bandgaps (shorter PL peak wavelengths) resulted in a lower yield, and is likely tied to the lower PL quantum efficiency that indicates a competitive carrier trapping mechanism. Overall, a maximum yield of 85% was obtained when the PL peak corresponds to 498 nm (Table 8). This observation illustrates that intentional band-tuning of perovskite NCs may activate previously non-reactive substrates.

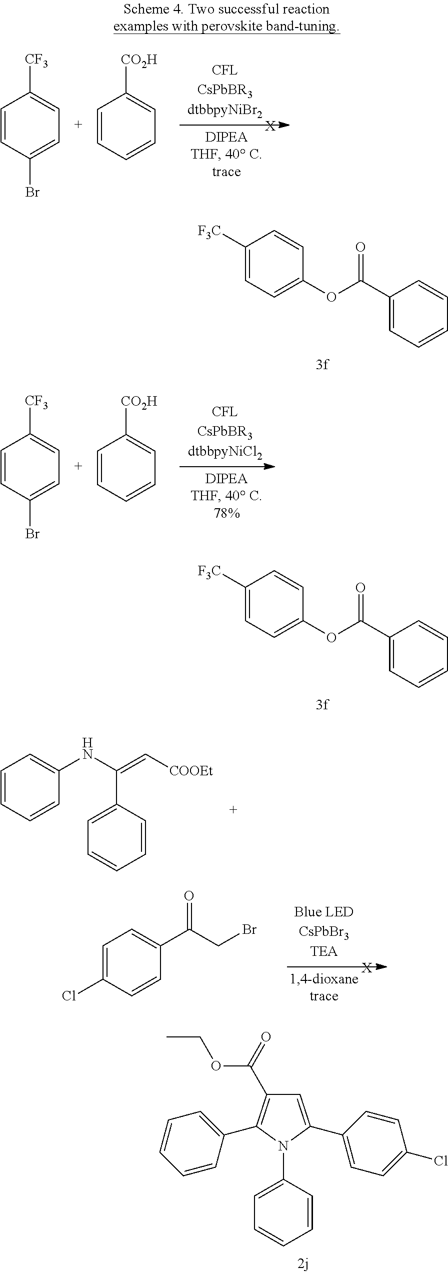

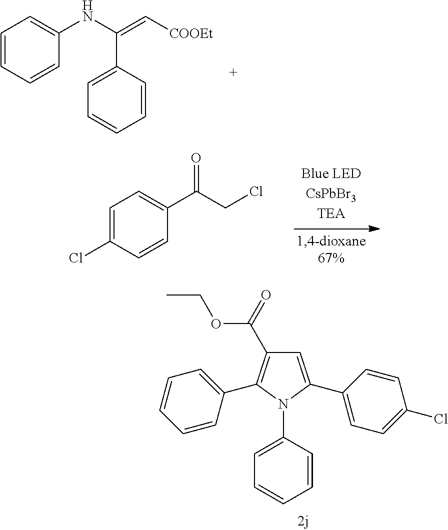

Furthermore, band-tuning may also result in an absolute discrepancy in photo-activation. It is widely accepted that the C—Cl bond are stronger than C—Br and hence harder to activate. Surprisingly, in reaction 2j in Scheme 4, α-chloroketone is observed to react to form 2j in dioxane in a yield of 67% while α-bromoketone is almost non-reactive at all. We assume that the band energy of CsPbBr₃ is not adequate to activate either Cl or Br-substrates in dioxane. However, ion-exchange may not only occur between CsPbBr₃ and CH₂Cl₂, but may also between CsPbBr₃ and suitable organic Cl-substrates. Interestingly, CsPbCl₃ was confirmed by XRD after reaction. Cl-substrate is the only Cl source and hence is activated in this type of reaction. Hence CB of photocatalyst is thus moved higher, simultaneously the reduction potential of the substrate moves in a reverse direction (FIG. 3b), overall making the ET possible and finally resulting in the pyrrole formation. This is also corroborating with the observation that, if reaction 2j was conducted in CH₂Cl₂, CsPbBr₃ NCs may first initiate ion-exchange with the solvent hence shift band energy, and finally catalyze both Br and Cl-substrates towards 2j formation. This result demonstrates that in-situ band-tuning of perovskite NCs may provide unexpected activity towards previously unachievable substrates.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Perovskite Preparation and Use

General Considerations. Commercial reagents were purchased from Sigma Aldrich and TCI America. Additionally, aldehydes were distilled prior to use. Tetrahydrofuran was distilled under N₂ over sodium benzophenoneketyl. All other solvents were purified by passage through columns of activated alumina. Two batches of CdSe Quantum Dots with nanoparticle concentration of 50 μmon in hexane with emission peak at 525 nm (particle size 2.8 nm) and 550 nm (particle size 3.5 nm) were purchased from Strem Chemicals. Silica gels (230-400 mesh) used for chromatography were purchased from Sorbent Technology. ¹H NMR and ¹³C NMR spectra were recorded in CDCl₃ on Bruker spectrometers at 400 or 500 (¹H NMR) and 100 or 125 MHz (¹³C NMR). All shifts are reported in parts per million (ppm) relative to residual CHCl₃ peak (7.27 and 77.2 ppm, ¹H NMR and ¹³C NMR, respectively). All coupling constants (J) are reported in hertz (Hz). Abbreviations are: s, singlet; d, doublet; t, triplet; q, quartet; brs, broad singlet. High-resolution mass spectra (HRMS) were measured on a 7T Bruker Daltonics FT-MS instrument. LC-MS spectra were measured on a Thermo Finnigan LTQ MS/MS with Agilent 1100 LC front end for MS with binary pump. TLC analysis was carried out on glass plates coated with silica gel 60 F254, 0.2 mm thickness. The plates were visualized using a 254 nm ultraviolet lamp or aqueous potassium permanganate solutions. ¹H NMR data are given for all compounds for characterization purposes. ¹H NMR, ¹³C NMR, and HRMS data are given for all new compounds.

A Shimadzu UV-2501 spectrophotometer was used to record the UV-vis absorption spectra in different solvents. A Horiba Fluoro-Max 4 fluorometer/phosphorometer was utilized to measure the steady-state emission spectra. Hitachi H-7500 transmission electron microscope was utilized to measure the TEM images. Philips Empyrean X-Ray Diffractometer was used to measure powder XRD.

Cyclic Voltammetry Measurement. The electrochemical experiments were carried out using a CHI 600E electrochemistry workstation (CHI, USA). A three-electrode cell was used with a Pt disc electrode as the working electrode, a Pt wire as the counter electrode and an Ag/AgCl electrode (Ag in 0.1 M AgNO₃ solution, from Sigma-Aldrich) as the reference electrode. Tetrabutylammonium hexafluorophosphate (0.1 M) was used as the supporting electrolyte. The potential values obtained in reference to Ag/AgCl were converted to the saturated calomel electrode (SCE) in order to directly compare with literature. All solutions were purged with N₂ for 20 min before experiments.

X-ray Crystallographic Analysis. Single crystals of 1d were obtained by slow diffusion of diethyl ether into dilute dichloromethane solution. A suitable crystal of 1d (CCDC 1889861) was selected and collected on a Bruker Apex Duo diffractometer with an Apex 2 CCD detector (Bruker, Madison, Wis., USA) at T=273 K, respectively. Mo radiation was used. The structure was processed with an Apex 2 v2010.9-1 software package (SAINT v.7.68A, XSHELL v.6.3.1). A direct method was used to solve the structure after multi-scan absorption corrections.

Synthesis of Perovskite CsPbBr₃ P1. CsPbBr₃ P1 NCs were synthesized by the modification of the method reported (J. Am. Chem. Soc. 2019, 141, 733). First, two precursor solutions are prepared in advance: 2.0 mmol CsBr dissolved in 2.0 mL H₂O and 2.0 mmol PbBr₂ dissolved in 3 mL DMF, respectively. Then, to a vigorously stirring mixture of 500 mL hexane, 8 mL oleic acid and 1.5 mL n-octylamine, the PbBr₂ DMF solution and CsBr solution are added dropwise. Along with mixing, an emulsion forms and the solution color turns from clear to slightly white. After that, acetone (400 mL) is added to break-up the emulsion. The CsPbBr₃ NCs are isolated by centrifugation at 2000 rpm for 2 min to discard large particles, and then 7000 rpm for 10 min to afford CsPbBr₃ P1.

Synthesis of CsPbBr₃ P1-oleyamine. Use the very similar method with the synthesis of Perovskite CsPbBr₃ P1, except the using of oleyamine instead of octylamine.

Colloidal CsPbBr₃ P2-P5. CsPbBr₃ P2-P5 NCs were synthesized according to the previous reported method (ACS Nano 2016, 10, 2071). First, Cs₂CO₃ (0.814 g) was loaded into 100 mL 3-neck flask along with octadecene (40 mL) and oleic acid (2.5 mL, OA), dried for 1 h at 120° C., and then heated under N₂ to 150° C. until all Cs₂CO₃ reacted with OA. Then, 5 mL ODE and PbBr₂ (0.069 g, 0.188 mmol) are loaded into 25 mL 3-neck flask and dried under vacuum for 1 h at 120° C. Dried oleylamine (0.5 mL) and dried OA (0.5 mL) were injected at 120° C. under N₂. After complete solubilization of PbBr₂, the temperature was raised to a desired value, and the prepared Cs-oleate solution (0.4 mL, 0.125 M in ODE) was quickly injected and, 5-10 s later, the reaction mixture was cooled by immersion in an ice-water bath. After centrifugation at 5000 rpm for 5 min to discard the precipitates, a bright yellow-green colloidal solution was obtained. The synthesized CsPbBr₃ are precipitated by adding 6 mL n-butanol and then centrifuged at 12000 rpm.

Synthesis of CsPbBr_{3-y}X_{y} (X═Cl, I). First, the colloidal CsPbBr₃ are prepared in CH₂Cl₂. Subsequently, the different volumes of trimethylsilyl chloride (TMSCl) or trimethylsilyl iodide (TMSI) DCM solution is dropped into the CsPbBr₃ solution until the desired emission peak position is achieved.

Photocatalytic organic synthesis procedure. In a typical synthesis, for instance 1a and 1b, to a 4 mL vial, CsPbBr₃ NCs P1 (1.0 mg), the corresponding bromide or chloride (0.5 mmol, 1.0 equiv.), 3-phenylpropanal (1.0 mmol, 2.0 equiv.), 2,6-lutidine (1.0 mmol, 2.0 equiv.), bis(2-chloroethyl)amine hydrochloride (0.1 mmol, 0.2 equiv.), and 2 mL CH₂Cl₂ were added and then stirred under the irradiation with a 12 W 455 nm Blue LED lamp, distance ˜8 cm. After 5-12 h, the mixture was poured into water, and extracted with CH₂Cl₂ (3×10 mL). The combined organic layers were washed with water, dried over Na₂SO₄ and concentrated in vacuo. The crude product was purified by column chromatography (silica gel, Hexane/EtOAc=10:1) to afford 1a or 1b.

### Example 2. Reaction Conditions

TABLE 2
Comparison of CsPbBr₃ P1 and other photocatalysts for the synthesis of 1a.^{a}

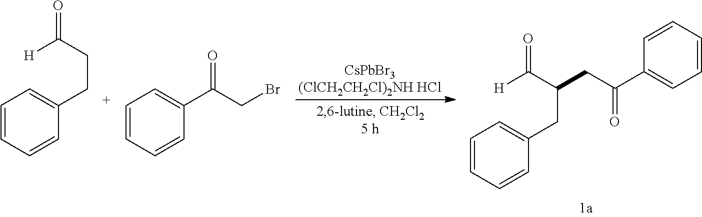

| Entry | Photocatalyst | Variation of conditions | Condition | Yield (%)^{b} |
|---|---|---|---|---|
| 1 | CsPbBr₃ | | N₂ | 85 |
| 2 | CsPbBr₃ | | air | 84 |
| 3 | CsPbBr₃ | 0.5 mg | air | 71 |
| 4 | CsPbBr₃ | 2.0 mg | air | 85 |
| 5 | 525 nm CdSe QDs | Hexane suspention | N₂ | 15 |
| 6 | 525 nm CdSe QDs | Hexane suspention | air | Trace |
| 7 | 525 nm CdSe QDs | Hexane suspention with oleic acid | N₂ | 53 |
| 8 | 525 nm CdSe QDs | Hexane suspention with oleic acid | air | <5 |
| 9 | 550 nm CdSe QDs | Hexane suspention | N₂ | 13 |
| 10 | 550 nm CdSe QDs | Hexane suspention | air | Trace |
| 11 | 550 nm CdSe QDs | Hexane suspention with oleic acid | N₂ | 31 |
| 12 | 550 nm CdSe QDs | Hexane suspention with oleic acid | air | Trace |
| 13 | 525 nm CdSe QDs | isolated powder | N₂ | Trace |
| 14 | 525 nm CdSe QDs | isolated powder | air | Trace |
| 15 | 550 nm CdSe QDs | isolated powder | N₂ | Trace |
| 16 | 550 nm CdSe QDs | isolated powder | air | Trace |
| 17 | Ir(ppy)₃ | | N₂ | 83 |
| 18 | Ir(ppy)₃ | | air | 79 |
| 19 | Ru(bpy)₃(PF₆)₂ | | N₂ | 83 |
| 20 | Ru(bpy)₃(PF₆)₂ | | air | <5 |
| 21 | DMPDP | | N₂ | 81 |
| 22 | DMPDP | | air | 18 |
| 23 | TiO₂ 32 nm | | N₂ | <5 |
| 24 | TiO₂ 32 nm | | air | <5 |
| 25 | PbBr₂ | | air | N.R. |
| 26 | PbBr₂ + CsBr (1:1 ratio) | | air | Trace |

^{a}Reaction conditions: 2-bromoacetophenone (0.5 mmol, 1.0 equiv.), octanal (1.0 mmol, 2.0 equiv.), Photocatalyst: CsPbBr₃ (1.0 mg), CdSe QDs (50 μl 50 μmol/L in hexanes), the other photocatalysts 1.0 mol %; (ClCH₂CH₂Cl)₂NH HCl (0.1 mmol, 20 mol %), 2,6-lutidine (1.0 mmol, 2.0 equiv.) and CH₂Cl₂ (1 mL) under 455 nm blue LED illumination at R.T.
^{b}Yield of 1a determined by ¹H NMR.

TABLE 3
Condition optimization for the synthesis of 1b.^{a}

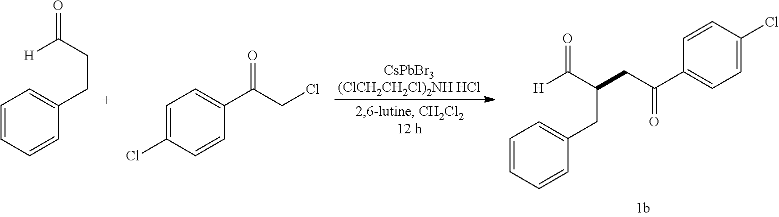 [SMILES: O=CCCc1ccccc1 ; O=C(CCl)c1ccc(Cl)cc1 ; O=C[C@@H](CC(=O)c1ccc(Cl)cc1)Cc1ccccc1]

| Entry | Variation of conditions | Yield (%)^{b} |
|---|---|---|
| 1 | None | 52 |
| 2 | Air-free | 50 |
| 3 | Bulk CsPbBr₃ ground powder instead of NCs | <5 |
| 4 | PbBr₂ instead of CsPbBr₃ NCs | Trace |
| 5 | PbBr₂ + CsBr (1:1 ratio) instead of CsPbBr₃ NCs | Trace |
| 6 | No CsPbBr₃ | Trace |
| 7 | No light | N.R. |

^{a}Reaction conditions: 2,4′-dichloroacetophenone (0.5 mmol, 1.0 equiv), octanal (1.0 mmol, 2.0 equiv), CsPbBr₃ (1.0 mg), (ClCH₂CH₂Cl)₂NH HC1 (0.1 mmol, 20 mol %), 2,6-lutidine (1.0 mmol, 2.0 equiv) and CH₂Cl₂ (1 mL) under 455 nm blue LED illumination at R.T.
^{b}Yield of 1b, determined by ¹H NMR.

TABLE 4
Condition optimization for the synthesis of 1c.^{a}

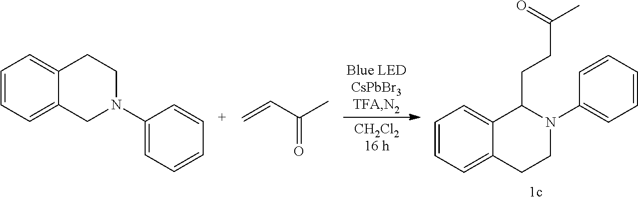

| Entry | Variation of conditions | Yield (%)^{b} |
|---|---|---|
| 1 | None | 90 |
| 2 | Air | 71 |
| 3 | ClCH₂CH₂Cl | 83 |
| 4 | THF | 65 |
| 5 | 1,4-dioxane | 59 |
| 6 | Capping ligand oleylamine instead of octylamine | 78 |
| 7 | No TFA | 26 |
| 8 | TFA 50 mol % | 56 |
| 9 | TFA 2 equiv. | 78 |
| 10 | TFA 5 equiv. | 35 |
| 11 | CsPbBr₃ 0.5 mg | 53 |
| 12 | CsPbBr₃ 2.0 mg | 89 |
| 13 | CsPbBr₃ ground powder instead of NCs | <5 |
| 14 | PbBr₂ instead of CsPbBr₃ NCs | N.R. |
| 15 | PbBr₂ + CsBr (1:1 ratio) instead of CsPbBr₃ NCs | <5 |
| 16 | Ru(bpy)₃(PF₆)₂ | 60 |
| 17 | Ir(ppy)₃ | N.R. |
| 18 | 525 nm CdSe QDs | N.R. |
| 19 | No CsPbBr₃ | N.R. |
| 20 | No light | N.R. |

^{a}Reaction conditions: 2-phenyl-1,2,3,4-tetrahydroisoquinoline (0.2 mmol, 1.0 equiv.), 3-buten-2-one (0.4 mmol, 2.0 equiv.), Photocatalyst: CsPbBr₃ (1.0 mg), CdSe QDs (50 μl 50 μmol/L in hexanes), the other photocatalysts 1.0 mol %; TFA (0.20 mmol, 1.0 equiv.), and CH₂Cl₂ (2 mL) under 455 nm blue LED illumination at R.T.
^{b}Yield of 1c determined by ¹H NMR.

TABLE 5
Condition optimization for the synthesis of 1d.^{a}

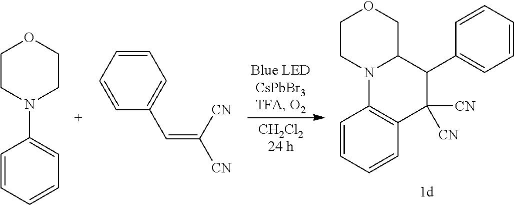

| Entry | Variation of conditions | Yield (%)^{b} |
|---|---|---|
| 1 | None | 79 |
| 2 | Air-free | <10 |
| 3 | Air | 26 |
| 4 | CsPbBr₃ 0.5 mg | 37 |
| 5 | CsPbBr₃ 1.0 mg | 73 |
| 6 | No TFA | 15 |
| 7 | TFA 50 mol % | 41 |
| 8 | TFA 2 equiv. | 63 |
| 9 | TFA 5 equiv. | 37 |
| 10 | Capping ligand oleylamine instead of octylamine | 75 |
| 11 | CsPbBr₃ ground powder instead of NCs | <5 |
| 12 | PbBr₂ instead of CsPbBr₃ NCs | N.R. |
| 13 | PbBr₂ + CsBr (1:1 ratio) instead of CsPbBr₃ NCs | <5 |
| 14 | Ru(bpy)₃(PF₆)₂ | 25 |
| 15 | Ir(ppy)₃ | N.R. |
| 16 | 525 nm CdSe QDs | N.R. |
| 17 | No CsPbBr₃ | N.R. |
| 18 | No light | N.R. |

^{a}Reaction conditions: 2-phenyl-1,2,3,4-tetrahydroisoquinoline (0.2 mmol, 1.0 equiv.), 3-Buten-2-one (0.4 mmo1, 2.0 equiv.), Photocatalyst: CsPbBr₃ (2.0 mg), CdSe QDs (50 μl 50 μmol/L in hexanes), the other photocatalysts (1 mol %), TFA (0.20 mmol, 1.0 equiv.), and CH₂Cl₂ (2 mL) under 455 nm blue LED illumination at R.T.
^{b}Yield of 1d determined by ¹H NMR.

TABLE 6
Optimization of reaction condition for the cyclization of benzaldehyde
phenylhydrazone with 2-bromoacetophenone.^{a}

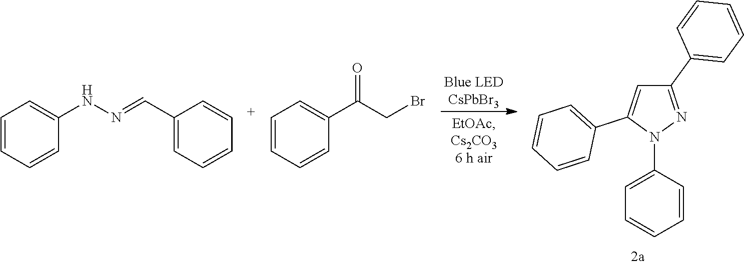

| Entry | Variation of conditions | Yield (%)^{b} |
|---|---|---|
| 1 | None | 86 |
| 2 | N₂-sparging for 15 min | 88 |
| 3 | CH₂Cl₂ instead of EtOAc | <5 |
| 4 | THF instead of EtOAc | 55 |
| 5 | CsPbBr₃ 0.5 mg | 53 |
| 6 | CsPbBr₃ 2.0 mg | 88 |
| 7 | MAPbBr₃ instead of CsPbBr₃ | 75 |
| 8 | Ir(PPY)₃ | 63 |
| 9 | Ir(ppy)₃, N₂-sparging for 15 min | 82 |
| 10 | Ru(bpy)₃(PF₆)₂ | <5 |
| 11 | Ru(bpy)₃(PF₆)₂, N₂-sparging for 15 min | 17 |
| 12 | CdSe QDs (525 nm) with oleic acid | N.R. |
| 13 | CdSe QDs (525 nm) with oleic acid, N₂- | N.R. |
| | sparging for 15 min | |
| 14 | TiO₂ 32 nm | N.R. |
| 15 | TiO₂ 32 nm N₂-sparging for 15 min | N.R. |
| 16 | No light | N.R. |
| 17 | No base | N.R. |
| 18 | No Perovskite | N.R. |

^{a}Reaction conditions: 1-benzylidene-2-phenylhydrazine (0.1 mmol, 1.0 equiv.), 2-bromoacetophenone (0.15 mmol, 1.5 equiv.), photocatalyst: CsPbBr₃ (1.0 mg); CdSe QDs (50 μl 50 μmol/L in hexanes), the other photocatalysts (1 mol %); base (1.0 equiv.) and solvent (2 mL) under 455 nm blue LED illumination at R.T. without N₂-sparging.
^{b}yield determined by ¹H NMR.

TABLE 7
Optimization of reaction condition for the cyclization of ethyl (Z)-3-phenyl-3-
(phenylamino)acrylate 2-bromoacetophenone.^{a}

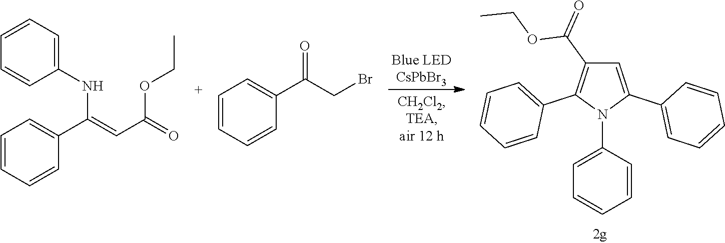

| Entry | Variation of conditions | Yield (%)^{b} |
|---|---|---|
| 1 | None | 93 |
| 2 | N₂-sparging for 15 min | 92 |
| 3 | 1,4-dioxane instead of CH₂Cl₂ | <5 |
| 4 | THF | <5 |
| 5 | MAPbBr₃ instead of CsPbBr₃ | 65 |
| 6 | CsPbBr₃ 0.5 mg | 72 |
| 7 | CsPbBr₃ 1.0 mg | 89 |
| 8 | Ir(PPY)₃ | 32 |
| 9 | Ir(ppy)₃, N₂-sparging for 15 min | 95 |
| 10 | Ru(bpy)₃(PF₆)₂ | 12 |
| 11 | Ru(bpy)₃(PF₆)₂, N₂-sparging for 15 min | 33 |
| 12 | CdSe QDs (525 nm) with oleic acid | N.R. |
| 13 | CdSe QDs (525 nm) with oleic acid, N₂- | 8 |
| | sparging for 15 min | |
| 14 | No light | N.R. |
| 15 | No base | 68 |
| 16 | No Perovskite | N.R. |

^{a}Reaction conditions: ethyl (Z)-3-phenyl-3-(phenylamino)acrylate (0.1 mmol, 1.0 equiv.), 2-bromoacetophenone (0.15 mmol, 1.5 equiv.), Photocatalyst: CsPbBr₃ (2.0 mg); CdSe QDs (50 μl 50 μmol/L in hexanes), the other photocatalysts (1 mol %); base (35 mol %) and solvent (2 mL) under 455 nm blue LED illumination at R.T. without N₂-sparging.
^{b}yield determined by ¹H NMR.

TABLE 8
Optimization of the coupling of benzoic acid with 4-bromotrifluorobenzene.^{a}

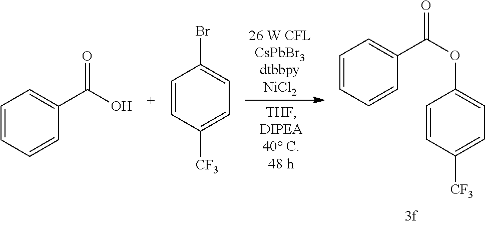 [SMILES: O=C(O)c1ccccc1 ; FC(F)(F)c1ccc(Br)cc1 ; O=C(Oc1ccc(C(F)(F)F)cc1)c1ccccc1]

| Entry | Variation of conditions | Yield (%)^{b} |
|---|---|---|
| 1 | None | 72 |
| 2 | N₂-sparging for 15 min | 78 |
| 3 | CsPbBr₃ 1.0 mg | 15 |
| 4 | CsPbBr₃ 3.0 mg | 56 |
| 5 | dttbpy NiBr₂ instead of dtbbpy NiCl₂ | <5 |
| 6 | CsPbBrₓCl₃₋ₓ (λ_{PL} = 498 nm) | 85 |
| 7 | CsPbBrₓCl₃₋ₓ (λ_{PL} = 465 nm) | 56 |
| 8 | CsPbBrₓCl₃₋ₓ (λ_{PL} = 413 nm) | <10 |
| 9 | CsPbBrₓI₃₋ₓ (λ_{PL} = 558 nm) | 69 |
| 10 | CsPbBrₓI₃₋ₓ (λ_{PL} = 600 nm) | 15 |
| 11 | Ir(ppy)₃ | 65 |
| 12 | Ir(ppy)₃, N₂-sparging for 15 min | 86 |
| 13 | Ru(bpy)₃(PF₆)₂ | N.R. |
| 14 | Ru(bpy)₃(PF₆)₂, N₂-sparging for 15 min | Trace |
| 15 | CdSe QDs (525 nm) with oleic acid | N.R. |
| 16 | CdSe QDs (525 nm) with oleic acid, N₂-sparging for 15 min | N.R. |
| 17 | No light | N.R. |
| 18 | No base | N.R. |
| 19 | No Perovskite | N.R. |
| 20 | No dtbbpy NiCl₂ | N.R. |

^{a}Performed with photocatalyst: CsPbBr₃ (5.0 mg); CdSe QDs (100 μl 50 μmol/L in hexanes), the other photocatalysts (2 mol %); Ni (5 mol %), benzoic acid (0.8 mmol, 2.0 equiv), 4-bromobenzotrifluoride (0.4 mmol, 1.0 equiv), and base (0.8 mmol, 2.0 equiv) under 26 W CFL for 48 h without N₂-sparging.
^{b}Yields determined by ¹H NMR.

TABLE 9
Reproducibility of the photocatalytic reactions with P1 in their corresponding optimized conditions.^{a}

| | Yield (%) ^{b} | | | | | |
|---|---|---|---|---|---|---|
| | 1a | 1c | 1d | 2a | 2g | 3f |
| 1^{st} | 85 | 90 | 79 | 86 | 93 | 72 |
| 2^{nd} | 82 | 81 | 80 | 82 | 79 | 67 |
| 3^{rd} | 86 | 86 | 71 | 84 | 84 | 71 |
| Average | 84 | 85 | 76 | 84 | 85 | 70 |

^{a}Conditions: Table 2, entry 2 for 1a; Table 4, entry 1 for 1c; Table 5, entry 1 for 1d; Table 6, entry 1 for 2a; Table 7, entry 1 for 2g; Table 8, entry 1 for 3f.
^{b} Yields determined by ¹H NMR.

TABLE 10
Key parameters for CsPbBr₃ P1-P5.^{a}

| | | | | | | | TON for 1a |
|---|---|---|---|---|---|---|---|
| | | | | Molecular | Surface area- | | (based on |
| | First exciton | E_{g}(d) | NC size | weight of NC | volume ratio | | molecular |
| NCs | peak (nm) | (eV) | d (nm) ^{b} | (g mol⁻¹) | (nm⁻¹) | Yield (%) ^{c} | weight) |
| P1 | | | 24 | 39,960,000 | 0.25 | 85 | 16,983,000 |
| P2 | 515 | 2.41 | 14.0 | 8,015,000 | 0.43 | 64 | 2,565,000 |
| P3 | 506 | 2.45 | 8.8 | 2,375,000 | 0.68 | 57 | 677,000 |
| P4 | 490 | 2.53 | 6.0 | 580,000 | 1.00 | 54 | 157,000 |
| P5 | 456 | 2.72 | 3.9 | 199,000 | 1.54 | 8 | 7900 |

^{a}The calculation method of key parameters.
^{b} Size of P1 was an estimated average size from TEM image.
^{c} Yields from Figure 6a.

### Example 3. Radical Trapping Experiments

### 1) The Trapping Experiment for the Synthesis of 1c

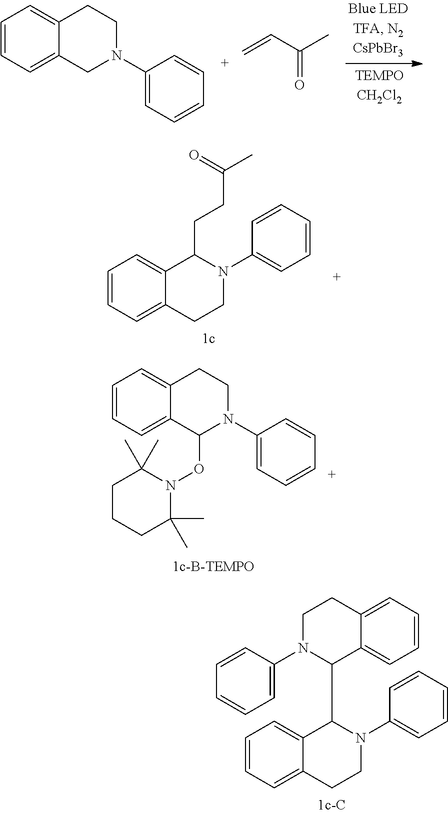

To a 4 mL vial, CsPbBr₃ NCs (2.0 mg), 2-phenyl-1,2,3,4-tetrahydroisoquinoline (0.25 mmol, 1.0 equiv.), 3-buten-2-one (0.5 mmol, 2.0 equiv.), TEMPO (0.5 mmol, 2.0 equiv.), trifluoroacetic acid (0.05 mmol, 20 mol %), and 2 mL DCM, the mixture was bubbled with N₂ for 10 min and then stirred. After irradiation with Blue LED for 8 h, trace amount of 1c and 1c-C was isolated, while 1d-B-TEMPO was detected by LC-MS. The crude self-coupling product 1c-C was purified by column chromatography to afford a mixture of diastereoisomers with 1.28:1 dr. ¹H NMR (500 MHz, CDCl₃) δ 7.39-6.86 (m, 16H, ArH (major+minor)), 6.79 (t, 2H, ArH (minor)), 6.75 (t, 2H, ArH (major)), 5.37 (s, 2H, CHCH, major), 5.34 (s, 2H, CHCH, minor), 3.56-3.52 (m, 2H, NCH₂CH₂, minor), 3.42-3.25 (m, 4H, NCH₂CH₂, major+minor), 2.87-2.82 (m, 2H, PhCH₂CH₂, major), 2.69-2.59 (m, 2H, PhCH₂CH₂, major+minor), 2.08-2.02 (m, 2H, PhCH₂CH₂, minor). ESI-MS m/z calcd for C₃₀H₂₉N₂⁺ ([M+H]⁺) 417.2331, found: 417.2335.

### 2) The Trapping Experiment for the Synthesis of 1d

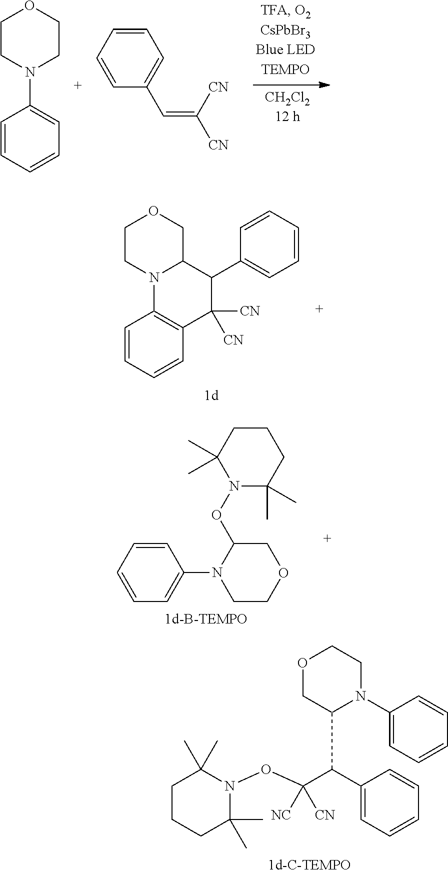

To an 8 mL vial, CsPbBr₃ NCs (2.0 mg), 4-phenylmorpholine (0.25 mmol, 1.0 equiv.), 2-benzylidenemalononitrile (0.5 mmol, 2.0 equiv.), trifluoroacetic acid (0.05 mmol, 20 mol %), and 1 mL DCM were added, the mixture was bubbled with 02 for 10 min and then stirred. After irradiation with Blue LED for 8 h, trace amount of 1d-B-TEMPO and 1d-C-TEMPO was detected by LC-MS.

### 3) The Trapping Experiment for the Synthesis of 2a

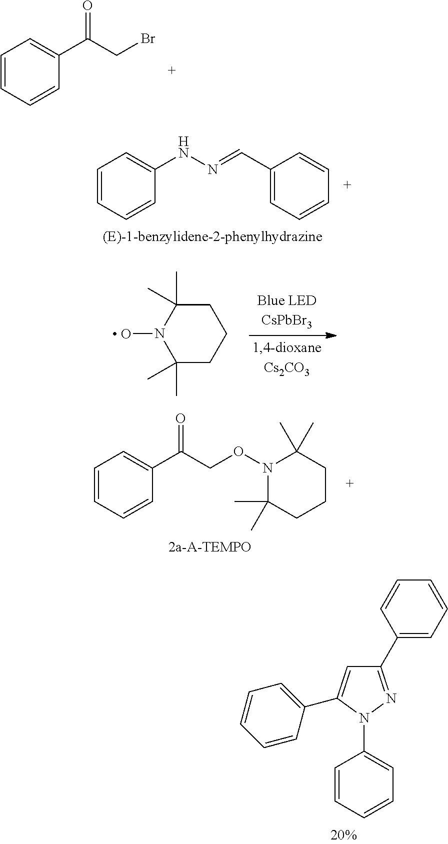

In a 4 mL vial equipped with (E)-1-benzylidene-2-phenylhydrazine (0.1 mmol), 2-bromoacetophenone (0.15 mmol), TEMPO (0.3 mmol), TEA (5 □L), CsPbBr₃ Perovskite NCs P1 (2.0 mg), and 1 mL DCM were added and then stirred under the irradiation with blue LED lamp for 24 h. Afford 2a with 20% yield, along the trapping product 2a-A-TEMPO was isolated and confirmed by ¹H NMR.

### 4) The Trapping Experiment of 2a-C

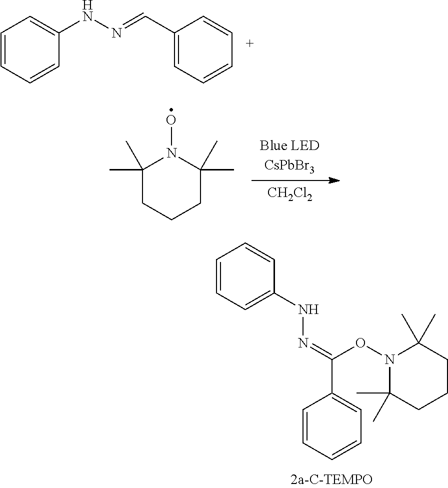

In a 4 mL vial equipped with (E)-1-benzylidene-2-phenylhydrazine (0.1 mmol), TEMPO (0.2 mmol), CsPbBr₃ Perovskite NCs P1 (2.0 mg), and 1 mL CH₂Cl₂ were added and then stirred under the irradiation with blue LED lamp for 24 h. The trapping products 2a-C-TEMPO was detected by LC-MS.

### 5) The Trapping Experiment for the Synthesis of 2g

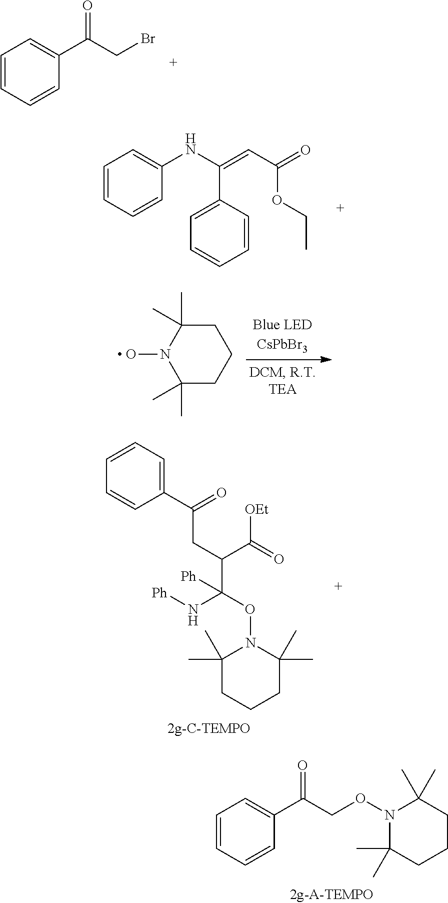

In a 4 mL vial equipped with ethyl (E)-3-phenyl-3-(phenylamino)acrylate (0.1 mmol), 2-bromoacetophenone (0.15 mmol), TEMPO (0.3 mmol), TEA (5 μL), CsPbBr₃ Perovskite NCs P1 (2.0 mg), and 1 mL DCM were added and then stirred under the irradiation with blue LED lamp for 24 h. Two trapping products 2g-A-TEMPO and 2g-C-TEMPO were detected by LC-MS.

### Example 4. Synthesis of Key Precursors

Substituted benzylidene phenylhydrazones and ethyl (Z)-3-phenyl-3-(phenylamino) acrylate were prepared according to literature procedures (Angew. Chem., Int. Ed. 2008, 47, 7230).

### Photocatalytic Organic Synthesis Procedure

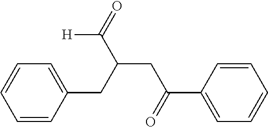 [SMILES: O=CC(CC(=O)c1ccccc1)Cc1ccccc1]

2-Benzyl-4-oxo-4-phenylbutanal (1a). The crude product was purified by column chromatography (silica gel, Hexane/EtOAc=10:1) to afford white solid, yield: 85%). ¹H NMR (500 MHz, CDCl₃) δ 9.93 (s, 1H), 7.93 (d, J=7.5 Hz, 2H), 7.59 (t, J=7.5 Hz, 2H), 7.47 (t, J=7.5 Hz, 2H), 7.34-7.22 (m, 5H), 3.47-3.41 (m, 2H), 3.20 (dd, J₁=14.0 Hz, J₂=6.0 Hz, 1H), 3.06-3.02 (m, 1H), 2.85 (dd, J₁=14.0 Hz, J₂=6.0 Hz, 1H).

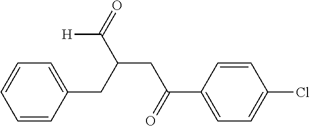 [SMILES: O=CC(CC(=O)c1ccc(Cl)cc1)Cc1ccccc1]

2-benzyl-4-(4-chlorophenyl)-4-oxobutanal (1b). The crude product was purified by column chromatography (silica gel, Hexane/EtOAc=10:1) to afford white solid, yield: 80% for 2-bromo-4′-chloroacetophenone as substrate; 52% for 2,4′-dichloro-acetophenone as substrate). ¹H NMR (400 MHz, CDCl₃) δ 9.89 (s, 1H), 7.83 (d, J=8.4 Hz, 2H), 7.41 (d, J=8.4 Hz, 2H), 7.31-7.28 (m, 2H), 7.26-7.18 (m, 3H), 3.42-3.34 (m, 2H), 3.17 (dd, J₁=14.0 Hz, J₂=6.0 Hz, 1H), 2.98-2.91 (m, 1H), 2.81 (dd, J₁=14.0 Hz, J₂=8.4 Hz, 1H). ¹³C NMR (100 MHz, CDCl₃) δ 202.80, 196.67, 139.79, 137.91, 134.72, 129.47, 128. 99, 128.92, 128.79, 16.81, 48.38, 37.05, 34.65. ESI-MS m/z calcd for C₁₇H₁₅ClO₂Na⁺ ([M+Na]⁺) 309.0658, found: 309.0672.

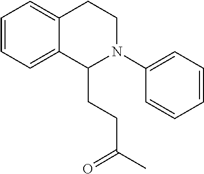 [SMILES: CC(=O)CCC1c2ccccc2CCN1c1ccccc1]

Synthesis of 4-(2-Phenyl-1,2,3,4-tetrahydroisoquinolin-1-yl)butan-2-one (1c). To a 4 mL vial, CsPbBr₃ NCs P1 (2.0 mg), 2-phenyl-1,2,3,4-tetrahydroisoquinoline (0.2 mmol, 1.0 equiv.), 3-buten-2-one (0.4 mmol, 2.0 equiv.), trifluoroacetic acid (0.20 mmol, 1.0 equiv.), and 2 mL DCM, the mixture was bubbled with N₂ for 10 min and then stirred under the irradiation with a 12 W 455 nm Blue LED lamp, distance ˜8 cm. After 16 h, the mixture was poured into water, and extracted with Et₂₀ (3×10 mL). The combined organic layers were washed with water, dried over Na₂SO₄ and concentrated in vacuo. The crude product was purified by column chromatography (silica gel, Hexane/EtOAc=10:1) to afford colorless oil 99 mg, yield: 90%). ¹H NMR (500 MHz, CDCl₃) δ 7.28-7.24 (m, 2H), 7.20-7.11 (m, 3H), 6.91 (d, J=8 Hz, 2H), 6.77 (t, J=7.5 Hz, 1H), 4.76 (dd, J₁=9.5 Hz, J₂=6.0 Hz, 1H), 3.68-3.55 (m, 2H), 3.06-3.00 (m, 1H), 2.78 (dt, J₁=16.5 Hz, J₂=4.5 Hz, 1H), 2.60 (t, J=7.0 Hz, 2H), 2.30-2.22 (m, 1H), 2.12 (s, 3H), 2.11-2.05 (m, 1H).

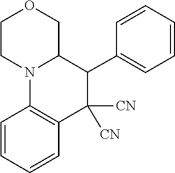 [SMILES: N#CC1(C#N)c2ccccc2N2CCOCC2C1c1ccccc1]

Synthesis of 5-Phenyl-1,2,4a,5-tetrahydro-[1,4]oxazino[4,3-a]quinoline-6,6(4H)-dicarbonitrile (1d). To an 8 mL vial, CsPbBr₃ NCs P1 (2.0 mg), 4-phenylmorpholine (0.20 mmol, 1.0 equiv.), 2-benzylidenemalononitrile (0.5 mmol, 2.0 equiv.), trifluoroacetic acid (0.20 mmol, 1.0 equiv., and 2 mL DCM were added, the mixture was bubbled with 02 for 10 min and then stirred under the irradiation with a 12 W 455 nm Blue LED lamp, distance ˜8 cm. After 24 h, the mixture was poured into water, and extracted with Et₂₀ (3×10 mL). The combined organic layers were washed with water, dried over Na₂SO₄ and concentrated in vacuo. The crude product was purified by column chromatography (silica gel, Hexane/EtOAc=10:1) to afford white solid, yield: 73%). ¹H NMR (500 MHz, CDCl₃) δ 7.56-7.45 (m, 6H), 7.40-7.36 (m, 1H), 6.97-6.84 (m, 2H), 4.03 (dd, J₁=14.0 Hz, J₂=4.5 Hz, 1H), 3.87 (td, J₁=13.0 Hz, J₂=4.0 Hz, 1H), 3.78 (d, J=16.0 Hz, 1H), 3.73-3.64 (m, 2H), 3.28 (d, J=13.5 Hz, 1H), 3.21-3.13 (m, 2H). Crystal Data for C₂₀H₁₉Cl_{0.2}N₃O (M=321.81 g mol⁻¹): monoclinic, space group P2₁/c (no. 14), a=20.198(3) Å, b=8.9832(15) Å, c=18.548(3) Å, β=109.622(5), V=3169.9(9) Å³, Z=8, T=273.15 K, μ(MoKα)=0.105 mm⁻¹, Dcalc=1.349 g cm⁻³, 82318 reflections measured (2.14°≤2θ≤61.996°), 10088 unique (Rᵢₙₜ=0.0289, R_{sigma}=0.0174) which were used in all calculations. The final R₁ was 0.0484 (I>2σ(I)) and wR₂ was 0.1382 (all data).

General procedure for the synthesis of pyrazoles (2a-2f). To a 4 mL vial, CsPbBr₃ Perovskite P1 (2.0 mg), benzylidene phenylhydrazone (0.1 mmol, 1 equiv.), the corresponding bromide (0.15 mmol, 1.5 equiv.), Cs₂CO₃ (0.15 mmol, 1.5 equiv.), and 2 mL 1,4-dioxane were added and then stirred under the irradiation with a 12 W 455 nm Blue LED lamp, distance ˜8 cm. After the reaction was complete (monitored via TLC, ˜6-12 h), the mixture was poured into water, and extracted with CH₂Cl₂ (3×10 mL). The combined organic layers were washed with water, dried over Na₂SO₄ and concentrated in vacuo. Purification of the crude product by column chromatography (silica gel) to afford the desired product.

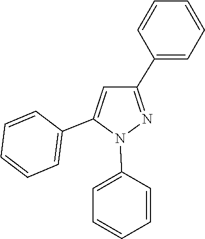 [SMILES: c1ccc(-c2cc(-c3ccccc3)n(-c3ccccc3)n2)cc1]

1,3,5-Triphenyl-1H-pyrazole (2a). The crude product was purified by column chromatography (silica gel, Hexane/EtOAc=20:1) to afford white solid: 25.4 mg, yield: 83%. ¹H NMR (500 MHz, CDCl₃) δ 7.95 (d, J=8.0 Hz, 2H), 7.45 (t, J=7.5 Hz, 2H), 7.41-7.29 (m, 11H), 6.86 (s, 1H).

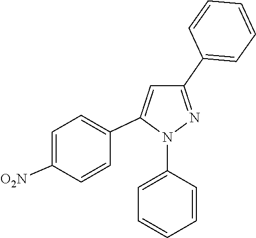 [SMILES: O=[N+]([O-])c1ccc(-c2cc(-c3ccccc3)nn2-c2ccccc2)cc1]

5-(4-nitrophenyl)-1,3-diphenyl-1H-pyrazole (2b). The crude product was purified by column chromatography (silica gel, Hexane/EtOAc=20:1) to afford white solid: 27.4 mg, yield: 84%. ¹H NMR (500 MHz, CDCl₃) δ 8.20 (d, J=7.0 Hz, 2H), 7.94 (d, J=8.0 Hz, 2H), 7.49-7.37 (m, 10H), 6.97 (s, 1H).

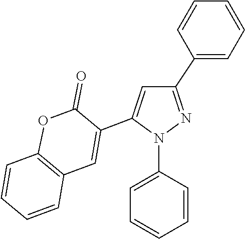 [SMILES: O=c1oc2ccccc2cc1-c1cc(-c2ccccc2)nn1-c1ccccc1]

3-(1,3-Diphenyl-1H-pyrazol-5-yl)-2H-chromen-2-one (2c). The crude product was purified by column chromatography (silica gel, Hexane/EtOAc=20:1) to afford yellow solid: 33.0 mg, yield: 89%. ¹H NMR (500 MHz, CDCl₃) δ 7.93 (d, J=8.0 Hz, 2H), 7.58-7.51 (m, 4H), 7.46-7.42 (m, 4H), 7.39-7.34 (m, 4H), 7.30-7.26 (m, 1H), 7.14 (s, 1H). ¹³C NMR (125 MHz, CDCl₃) δ 161.50, 156.30, 154.80, 145.15, 145.08, 142.93, 139.77, 135.37, 134.97, 132.02, 131.31, 130.80, 130.66, 128.57, 128.51, 121.38, 121.38, 121.24, 110.37, 110.32. ESI-MS m/z calcd for C₂₄H₁₇N₂O₂⁺ ([M+H]⁺) 365.1290, found: 365.1276.

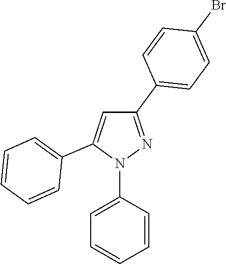 [SMILES: Brc1ccc(-c2cc(-c3ccccc3)n(-c3ccccc3)n2)cc1]

3-(4-Bromophenyl)-1,5-diphenyl-1H-pyrazole (2d). The crude product was purified by column chromatography (silica gel, Hexane/EtOAc=20:1) to afford white solid: 31.2 mg, yield: 83%. ¹H NMR (500 MHz, CDCl₃) δ 7.82 (d, J=8.5 Hz, 2H), 7.58 (d, J=8.5 Hz, 2H), 7.39-7.29 (m, 10H), 6.82 (s, 1H).

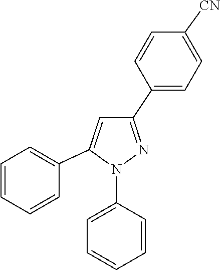 [SMILES: N#Cc1ccc(-c2cc(-c3ccccc3)n(-c3ccccc3)n2)cc1]

4-(1,5-Diphenyl-1H-pyrazol-3-yl)benzonitrile (2e). The crude product was purified by column chromatography (silica gel, Hexane/EtOAc=20:1) to afford yellow solid: 28.8 mg, yield: 90%. ¹H NMR (500 MHz, CDCl₃) δ 8.05 (d, J=8.0 Hz, 2H), 7.70 (d, J=8.0 Hz, 2H), 7.40-7.36 (m, 8H), 7.36-7.29 (m, 2H), 6.89 (s, 1H).

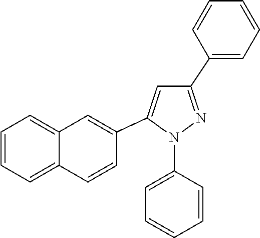 [SMILES: c1ccc(-c2cc(-c3ccc4ccccc4c3)n(-c3ccccc3)n2)cc1]

5-(Naphthalen-2-yl)-1,3-diphenyl-1H-pyrazole (2f). The crude product was purified by column chromatography (silica gel, Hexane/EtOAc=20:1) to afford white solid: 28.0 mg, yield: 82%. ¹H NMR (500 MHz, CDCl₃) δ 7.99 (d, J=8.0 Hz, 2H), 7.89 (s, 1H), 7.86-7.77 (m, 3H), 7.54-7.44 (m, 6H), 7.38-7.31 (m, 5H), 6.97 (s, 1H).

General procedure for the synthesis of pyrroles (2g-2i). To a 4 mL vial, CsPbBr₃ Perovskite P1 (2.0 mg), Ethyl (Z)-3-phenyl-3-(phenylamino)acrylate (0.1 mmol, 1.0 equiv), the corresponding bromide (0.15 mmol, 1.5 equiv), TEA (0.035 mmol, 35 mol %), and 2 mL CH₂Cl₂ were added and then stirred under the irradiation with a 12 W 455 nm Blue LED lamp, distance ˜8 cm. After the reaction was complete (monitored via TLC), the mixture was poured into water, and extracted with CH₂Cl₂ (3×10 mL). The combined organic layers were washed with water, dried over Na₂SO₄ and concentrated in vacuo. Purification of the crude product by column chromatography (silica gel) to afford the desired product.

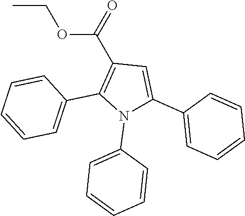 [SMILES: CCOC(=O)c1cc(-c2ccccc2)n(-c2ccccc2)c1-c1ccccc1]

Ethyl 1,2,5-triphenyl-1H-pyrrole-3-carboxylate (2g). White solid: 35.0 mg, yield: 95%. ¹H NMR (500 MHz, CDCl₃) δ 7.24-7.17 (m, 11H), 7.12-7.10 (m, 2H), 6.95 (s, 1H), 6.93-6.90 (m, 2H), 4.17 (q, J=7.0 Hz, 2H), 1.17 (t, J=7.0 Hz, 3H).

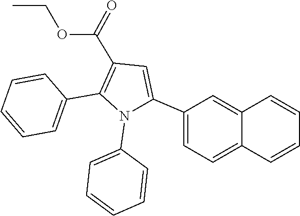 [SMILES: CCOC(=O)c1cc(-c2ccc3ccccc3c2)n(-c2ccccc2)c1-c1ccccc1]

Ethyl 5-(naphthalen-2-yl)-1,2-diphenyl-1H-pyrrole-3-carboxylate (2h). The crude product was purified by column chromatography (silica gel, Hexane/EtOAc=10:1) to afford white solid: 32.7 mg, yield: 78%. ¹H NMR (500 MHz, CDCl₃) δ 7.76 (m, 1H), 7.75-7.61 (m, 3H), 7.45-7.43 (m, 2H), 7.25-7.15 (m, 8H), 7.09 (s, 1H), 7.00-6.98 (m, 2H), 4.21 (q, J=7.0 Hz, 2H), 1.22 (t, J=7.0 Hz, 3H).

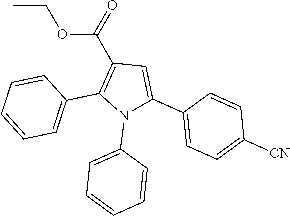 [SMILES: CCOC(=O)c1cc(-c2ccc(C#N)cc2)n(-c2ccccc2)c1-c1ccccc1]

Ethyl 5-(4-cyanophenyl)-1,2-diphenyl-1H-pyrrole-3-carboxylate (2i). The crude product was purified by column chromatography (silica gel, Hexane/EtOAc=10:1) to afford white solid: 24.7 mg, yield: 63%. ¹H NMR (500 MHz, CDCl₃) δ 7.47 (d, J=8.0 Hz, 1H), 7.28-7.22 (m, 6H), 7.20-7.16 (m, 4H), 7.10 (s, 1H), 6.94 (d, J=8.0 Hz, 2H), 4.20 (q, J=7.0 Hz, 2H), 1.19 (t, J=7.0 Hz, 3H).

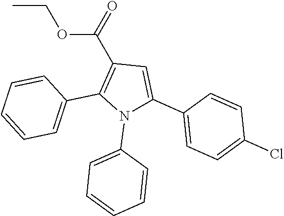 [SMILES: CCOC(=O)c1cc(-c2ccc(Cl)cc2)n(-c2ccccc2)c1-c1ccccc1]

Ethyl 5-(4-chlorophenyl)-1,2-diphenyl-1H-pyrrole-3-carboxylate (2j). To a 4 mL vial, CsPbBr₃ P1 (3.0 mg), ethyl (Z)-3-phenyl-3-(phenylamino)acrylate (0.1 mmol, 1.0 equiv.), 2,4′-dichloroacetophenone (0.15 mmol, 1.5 equiv.), TEA (0.035 mmol, 35 mol %), and 2 mL 1,4-dioxane were added and then stirred under the irradiation with a 12 W 455 nm blue LED lamp, distance ˜8 cm. After irradiation for 24 h, the mixture was poured into water, and extracted with CH₂Cl₂ (3×10 mL). The combined organic layers were washed with water, dried over Na₂SO₄ and concentrated in vacuo. Purification of the crude product by column chromatography (silica gel, Hexane/EtOAc=10:1) to afford white solid: 26.7 mg, yield: 67%. ¹H NMR (400 MHz, CDCl₃) δ 7.23-7.12 (m, 10H), 7.01-6.98 (m, 2H), 6.94 (s, 1H), 6.92-6.89 (m, 2H), 4.18 (q, J=7.2 Hz, 2H), 1.71 (t, J=7.2 Hz, 3H). ¹³C NMR (100 MHz, CDCl₃) δ 164.58, 140.12, 137.64, 133.42, 132.76, 131.43, 131.23, 130.67, 129.69, 128.75, 128.74, 128.68, 128.27, 128.24, 127.82, 127.79, 127.25, 114.41, 111.11, 59.67, 14.18. ESI-MS m/z calcd for C₂₅H₂₁ClNO₂⁺ ([M+1]⁺) 402.1261, found: 402.1266.

General procedure of the couplings of carboxylic acid and aryl bromides (3a-3f). To a 4 mL vial, 5.0 mg CsPbBr₃ P1 carboxylic acid (0.8 mmol, 2.0 equiv), aryl bromide (0.4 mmol, 1.0 equiv), dtbbpy.Ni(II)Cl₂ (8.0 mg, 0.02 mmol, 0.05 equiv), and DIPEA (139 □L, 0.8 mmol, 2.0 equiv) were added into 2 mL THF. The mixture was stirred at 40° C. under the irradiation with two 26 W compact fluorescent lights, distance ˜5 cm. After 48 h, the mixture was poured into water, and extracted with EtOAc (3×10 mL). The combined organic layers were washed with water, dried over Na₂SO₄ and concentrated in vacuo. Purification of the crude product by column chromatography (silica gel) to afford the desired product.

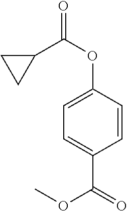 [SMILES: COC(=O)c1ccc(OC(=O)C2CC2)cc1]

Methyl 4-((cyclopropanecarbonyl)oxy)benzoate (3a). The crude product was purified by column chromatography (silica gel, Hexane/EtOAc=20:1) to afford 66 mg white solid, yield: 72%. ¹H NMR (500 MHz, CDCl₃) δ 8.08 (d, J=8.5 Hz, 2H), 7.20 (d, J=9.0 Hz, 2H), 3.93 (s, 3H), 1.89-1.85 (m, 1H), 1.22-1.19 (m, 2H), 1.09-1.05 (m, 2H). The characterization data are consistent with those reported in the literature.

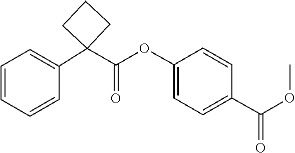 [SMILES: COC(=O)c1ccc(OC(=O)C2(c3ccccc3)CCC2)cc1]

Methyl 4-((1-phenylcyclobutane-1-carbonyl)oxy)benzoate (3b). The crude product was purified by column chromatography (silica gel, Hexane/EtOAc=10:1) to afford white solid, yield: 74%. ¹H NMR (500 MHz, CDCl₃) δ 8.04 (d, J=8.5 Hz, 2H), 7.45-7.40 (m, 4H), 7.32 (t, J=6.5 Hz, 2H), 7.05 (d, J=9.0 Hz, 2H), 3.92 (s, 3H), 3.05-2.99 (m, 2H), 2.71-2.65 (m, 2H), 2.21-2.14 (m, 1H), 2.04-1.97 (m, 1H). ¹³C NMR (125 MHz, CDCl₃) δ 173.88, 166.33, 154.77, 142.65, 131.05, 128.54, 127.56, 127.03, 126.35, 121.35, 52.67, 52.18, 32.26, 16.68. ESI-MS m/z calcd for C₁₉H₁₈O₄Na⁺ ([M+Na]⁺) 333.1103, found: 333.1135.

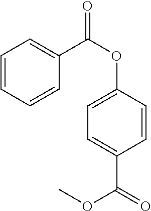 [SMILES: COC(=O)c1ccc(OC(=O)c2ccccc2)cc1]

Methyl 4-(benzoyloxy)benzoate (3c). The crude product was purified by column chromatography (silica gel, Hexane/EtOAc=10:1) to afford white solid, yield: 76%. ¹H NMR (500 MHz, CDCl₃) δ 8.23 (d, J=8.0 Hz, 2H), 8.15 (d, J=8.5 Hz, 2H), 7.68 (t, J=7.5 Hz, 1H), 7.55 (t, J=7.5 Hz, 2H), 7.33 (d, J=8.5 Hz, 2H), 3.96 (s, 3H).

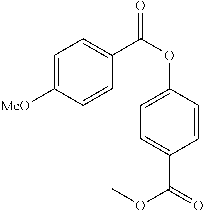 [SMILES: COC(=O)c1ccc(OC(=O)c2ccc(OC)cc2)cc1]

4-(Methoxycarbonyl)phenyl 4-methoxybenzoate (3d). The crude product was purified by column chromatography (silica gel, Hexane/EtOAc=10:1) to afford white solid, yield: 69%. ¹H NMR (500 MHz, CDCl₃) δ 8.17 (d, J=9.0 Hz, 2H), 8.14 (d, J=9.0 Hz, 2H), 7.32 (d, J=8.5 Hz, 2H), 7.02 (d, J=8.5 Hz, 2H), 3.95 (s, 3H), 3.93-3.95 (s, 3H).

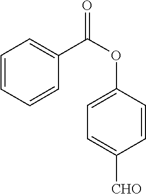 [SMILES: O=Cc1ccc(OC(=O)c2ccccc2)cc1]

4-Formylphenyl benzoate (3e). The crude product was purified by column chromatography (silica gel, Hexane/EtOAc=20:1) to afford white solid, yield: 75%. ¹H NMR (500 MHz, CDCl₃) δ 10.02 (s, 1H), 8.21 (d, J=7.0 Hz, 2H), 7.97 (d, J=8.5 Hz, 2H), 7.66 (t, J=7.5 Hz, 1H), 7.53 (t, J=8.0 Hz, 2H), 7.41 (d, J=8.5 Hz, 2H).

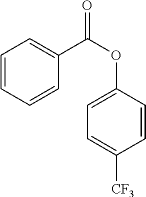 [SMILES: O=C(Oc1ccc(C(F)(F)F)cc1)c1ccccc1]

4-(Trifluoromethyl)phenyl benzoate (3f). The crude product was purified by column chromatography (silica gel, Hexane/EtOAc=20:1) to afford 89 mg white solid, yield: 84%. ¹H NMR (500 MHz, CDCl₃) δ 8.24 (d, J=7.0 Hz, 2H), 7.73 (d, J=9.0 Hz, 2H), 7.70 (t, J=7.5 Hz, 1H), 7.56 (t, J=7.5 Hz, 2H), 7.39 (d, J=8.5 Hz, 2H).

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims. All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for photo-catalytic synthesis of an organic molecule comprising:
a) contacting a lead halide perovskite, first redox substrate, second redox substrate, and solvent to form a mixture, wherein:
i) the first redox substrate comprises an alkylamine, and the second redox substrate comprises an alpha, beta-unsaturated carbonyl or alpha, beta-unsaturated nitrile, wherein a carbon-carbon (C—C) bond is formed via carbon-hydrogen activation; or
ii) the first redox substrate comprises an imine or enamine, and the second redox substrate comprises an alpha-halocarbonyl, wherein a carbon-nitrogen (C—N) bond is formed via N-heterocyclization; or
iii) the first redox substrate comprises an enamine or haloaryl, and the second redox substrate comprises an alpha-halocarbonyl or carboxylic acid, wherein a carbon-oxygen (C—O) bond is formed via cross-coupling; and
b) irradiating the mixture at a suitable wavelength to form at least one covalent bond between the first redox substrate and second redox substrate;
wherein the perovskite photo-catalyzes formation of the C—C, C—N, or C—O bond and the organic molecule is thereby synthesized.

2. The method of claim 1 wherein the perovskite is APbBr₃ wherein A is an alkali metal.

3. The method of claim 1 wherein the perovskite is CsPbBr₃.

4. The method of claim 1 wherein the perovskite is pretreated with a trialkylsilylhalide.

5. The method of claim 1 wherein the perovskite is CsPbBr_{3-y}X_{y} wherein X is Cl or I, and y is 1-3.

6. The method of claim 1 wherein the perovskite is in the form of a nanocrystal or colloid.

7. The method of claim 1 wherein the perovskite has an average particle size of about 1 nanometer to about 150 nanometers.

8. The method of claim 1 wherein the alkylamine is an N-aryl substituted nitrogen heterocycloalkyl.

9. The method of claim 1 wherein the enamine is an alkylidene hydrazine.

10. The method of claim 1 wherein the suitable wavelength is provided by a blue light emitting diode or compact fluorescent light bulb.

11. The method of claim 1 wherein the suitable wavelength is about 355 nanometers to about 465 nanometers.

12. The method of claim 1 wherein the solvent is dichloromethane, ethyl acetate, tetrahydrofuran, dioxane, hexanes, or toluene.

13. The method of claim 1 wherein the perovskite photo-catalyzes formation of the C—C bond.

14. The method of claim 13 wherein the mixture is contacting oxygen.

15. The method of claim 14 wherein the mixture is contacting an acidic additive.

16. The method of claim 1 wherein the perovskite photo-catalyzes formation of the C—N bond.

17. The method of claim 16 wherein the mixture is contacting a basic additive and air.

18. The method of claim 1 wherein the perovskite photo-catalyzes formation of the C—O bond.

19. The method of claim 18 wherein the mixture is contacting a transition metal co-catalyst and a basic additive.

20. The method of claim 19 wherein the co-catalyst is a nickel catalyst.
